(19) United States
(12) Patent Application Publication
(10) Pub. No.: US 2015/0177247 A1
(43) Pub. Date: Jun. 25, 2015

(54) METHOD FOR IDENTIFYING MARKER SEQUENCES FOR GYNAECOLOGICAL MALIGNANT TUMOURS

(71) Applicant: Protagen AG, Dortmund (DE)

(72) Inventors: ANGELIKA LÜKING, Bochum (DE); Axel Kowald, Bochum (DE); Annabel Höpner, Bielefeld (DE); Peter Schulz-Knappe, Hemmingen (DE); Christian Scheer, Dortmund (DE); Heidelinde Fiegl, Ellbogen (AT); Günter Daxenbichler, Innsbruck (AT)

(21) Appl. No.: 14/633,773

(22) Filed: Feb. 27, 2015

Related U.S. Application Data
(63) Continuation of application No. 14/365,413.

(30) Foreign Application Priority Data
Dec. 16, 2011 (EP) 11194099.5

(51) Int. Cl. G01N 33/574; C07K 14/47

(52) U.S. Cl. CPC G01N 33/57411; C07K 14/47; G01N 2570/00

(57) ABSTRACT

The present invention relates to a method for identifying marker sequences for gynaecological malignoma, the marker sequences identified with the aid of this method and diagnostic use thereof, diagnostic devices containing marker sequences for gynaecological malignoma, in particular an arrangement and a protein array, and use thereof. The invention also relates to method for the screening of potential active agents for the treatment and prevention of gynaecological malignoma by means of these marker sequences.

The present invention relates to a method for identifying marker sequences for gynaecological malignoma, the marker sequences identified with the aid of this method and diagnostic use thereof, diagnostic devices containing marker sequences for gynaecological malignoma, in particular an arrangement and a protein array, and use thereof. The invention also relates to methods for screening potential active agents for the treatment and prevention of gynaecological malignoma by means of these marker sequences. Protein arrays are gaining increasing industrial importance in analysis and diagnosis as well as in pharmaceutical development. Protein arrays have become established as screening tools.

Here, the rapid and highly parallel detection of a multiplicity of specifically binding analysis molecules in a single experiment is made possible. To produce protein arrays, it is necessary to have the required proteins available. In particular, protein expression libraries have been established for this purpose. High-throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is closely linked to the progress of the genome sequencing projects and the annotation of these gene sequences. In addition, the determination of the expressed sequence is not always clear due to differential splicing processes.

This problem can be avoided by the use of cDNA expression libraries (Bssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). Here, the cDNA of a specific tissue is cloned into a bacterial or eukaryotic expression vector, such as yeast. The vectors used for the expression are generally characterised in that they carry inducible promoters that may be used to control the time of protein expression. In addition, expression vectors have sequences for what are known as affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

By way of example, the gene products of a cDNA expression library from human foetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from expression libraries could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Bussow (2000) supra; Lueking, A., Horn, M., Eickhoff, H., Bissow, K., Lehrach, H. and Walter, G. (1999) Protein microarrays for gene expression and antibody screening. Analytical Biochemistry, 270, 103-111). Such protein biochips based on cDNA expression libraries are disclosed in particular in WO 99/57311 and WO 99/5731.2.

Furthermore, in addition to antigen-presenting protein arrays, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

Cervical carcinoma (Carcinoma cervicis uteri), also referred to as collum carcinoma or cervical cancer, is an aggressive (malignant) tumour of the cervix (Cervix uteri). Globally, it is the second most common aggressive tumour in women. Histologically, it is a squamous cell carcinoma in the majority of cases. The most frequent cause for a cervical carcinoma is an infection with certain types of the human papilloma virus (HPV). The cervical carcinoma initially causes no pain, just occasional slight spotting. Only when the tumour is larger and degrades with ulcer formation is there a flesh-coloured, sweet-smelling discharge. In the early stage, the complete removal of the change by means of conisation is sufficient. In the advanced stage, the removal of the entire cervix with surrounding tissue and sometimes also further organs is necessary.

In the global cause of death statistics of gynaecological malignomas, the (invasive) cervical carcinoma growing into the surrounding tissue therefore occupies first place in particular, with a mortality rate (lethality) of more than 60%. In Germany, approximately more than 6,000 women newly contract cervical carcinoma every year, and approximately 1,800 die as a result of this disease. The likelihood of survival of patients after a period of 5 years is approximately 60%.

Cervical carcinoma is diagnosed most frequently in the age range from 45 to 55 years, however preliminary stages may already occur in patients aged from 20 to 30 years. The average age at initial diagnosis of cervical carcinoma has decreased in the last 25 years by 14 years and is currently at approximately 52 years. A peak between the 35^{th} and 54^{th} year of life can be observed in the age distribution as well as a further rise from the 65^{th} year of life. In 2003, the frequency of the disease demonstrated an altered age distribution because the diagnosis was made much more frequently in women aged between 25 and 35 years than in women aged above 65 years old. The disease may also occur during pregnancy. Here, the incidence is 1.2 per 10,000 pregnancies.

It is assumed that a large proportion of cervical carcinomas are caused by the human papilloma virus (HPV). A test for early detection is constituted by the pap test. However, cell changes that constitute a preliminary stage for a cancer disease or even subsequently a carcinoma develop only in 2 to 8 percent of HIV-infected women.

The diagnosis of a cervical carcinoma can only be made by histological examination of tissue pieces. These are either obtained through a selective sample removal from an area at the cervix abnormal during a colposcopy, a conisation following repeatedly abnormal pap test, or a scraping in the event of suspicion of a change in the cervical canal.

Michael E. Hudson et al. (PNAS (2007) vol. 104, Nr. 44, pages 17494-17499) describes the identification of markers for cervical carcinoma with the aid of protein microarrays. Here, the sera of cancer patients are compared with those of healthy subjects in order to identify proteins that are expressed differently. It was found that 49 proteins are expressed differently in the tissue of cancer patients compared with the healthy subjects, that is to say, with the aid of this study, relevant markers referred to as tumour-associated autoantigens were identified in the tissue of the patients. However, no markers for the detection of these differently expressed proteins or the tumour-associated autoantigens in the serum were provided with this study (see page 17498, column 2, penultimate paragraph).

Karen S. Anderson et al. (Journal of Proteom Research (2011) vol. 10, Nr. 1, pages 85-96) discloses the detection of autoantibodies against tumour-associated proteins with the aid of protein microarrays. The protein microarrays used here are produced in that full-length clones of the cDNAs coding for potentially tumour-associated antigens are printed onto the support, expressed and then tested comparatively with the sera of female breast cancer patients and control individuals.

US 2005/221342 A1 discloses SEQ ID. No. 538 of the present invention and generally also the use of this sequence, but not the specific application with gynaecological malignoma.

In the case of gynaecological malignomas, in particular cervical carcinoma, an early diagnosis is key for the further progression of the disease and for the prognosis.

There is thus a need for indication-specific diagnostic devices and methods for gynaecological malignoma, in particular for cervical carcinoma. The object of the present invention is to provide improved means for the early detection and therapy control in the case of gynaecological malignoma.

The invention relates to a method for identifying marker sequences for gynaecological malignoma, characterised in that
a. marker sequence candidates for gynaecological malignoma are identified in that a support, on which at least 1,000 different proteins are immobilised, is brought into contact with a serum sample from a female patient with gynaecological malignoma and proteins are identified that demonstrate an interaction with the serum (marker sequence candidates), and
b. the interaction of one or more marker sequence candidates from a. with the serum of female patients with gynaecological malignoma is determined compared with
   the interaction of the marker sequence candidate(s) from a. with the serum of female patients with benign changes and
   the interaction of the marker sequence candidate(s) from a. with the serum of healthy control individuals, and
c. marker sequences are identified in that they demonstrate an interaction with the serum from female patients with gynaecological malignoma that is different compared with the interaction with the serum from female patients with benign changes and the serum of healthy control individuals.

For example, the comparative evaluation of the data concerning the interaction from b. is performed by means of statistical analysis, for example as described in the examples.

With the aid of this method, marker sequences for gynaecological malignoma can be identified that are highly specific. Marker sequences that are found with this method on the one hand enable the early detection of gynaecological malignoma, for example of preliminary stages thereof, and on the other hand enable the distinction of gynaecological malignoma or preliminary stages thereof from benign changes. An early diagnosis and optionally a targeted treatment and also a considerably improved prognosis are thus possible. In contrast to the experiments described in the prior art by Hudson et al. (2007, above) and Anderson et al. (2011, above), marker sequences that are more specific, for example because they are also suitable for discrimination of gynaecological malignoma from non-malignant changes of the tissue (benign change), are identified with the aid of the method according to the invention. Furthermore, the marker sequences identifiable with the aid of the method according to the invention are suitable not only for testing tissue sections or biopsy material from female patients, but also for the analysis of bodily fluids, such as serum. A quick and cost-effective use or application of the marker sequences according to the invention is thus possible.

The invention also relates to the marker sequences for gynaecological malignoma identified with the method according to the invention. The invention relates to marker sequences for gynaecological malignoma obtainable by a method according to the invention and selected from the sequences comprising SEQ ID No. 1-1467 and partial sequences of SEQ ID No. 1-1467 with at least 90%, preferably 95%, of the length of the sequences SEQ ID No. 1-1467, and homologues of SEQ ID No. 1-1467 and partial sequences thereof with an identity of at least 95%, preferably 98% or more, to the corresponding nucleic acid and/or protein sequences, and sequences coded by SEQ ID No. 1-489, partial sequences thereof and homologues.

The invention also relates to an arrangement comprising one or more marker sequences according to the invention.

The invention also relates to a protein array comprising one or more marker sequences according to the invention.

The invention also relates to a diagnostic tool comprising one or more marker sequences according to the invention and optionally further additives and/or excipients.

The invention also relates to a test kit comprising one or more marker sequences according to the invention and optionally further additives and/or excipients.

The invention also relates to an arrangement according to the invention, characterised in that 2 or 3, preferably 4 or 5, particularly preferably 7 or 8 or more, different marker sequences for gynaecological malignoma are used simultaneously.

The invention also relates to a protein array according to the invention, characterised in that 2 or 3, preferably 4 or 5, particularly preferably 7 or 8 or more, different marker sequences for gynaecological malignoma are used simultaneously.

The invention also relates to a diagnostic tool according to the invention, characterised in that 2 or 3, preferably 4 or 5, particularly preferably 7 or 8 or more, different marker sequences for gynaecological malignoma are used simultaneously.

The invention also relates to a test kit according to the invention, characterised in that 2 or 3, preferably 4 or 5, particularly preferably 7 or 8 or more, different marker sequences for gynaecological malignoma are used simultaneously.

The invention also relates to the use of one or more marker sequences according to the invention, an arrangement according to the invention, a protein array according to the invention, a diagnostic tool according to the invention or a test kit according to the invention for the early detection, diagnosis, prognosis, therapy control and/or aftercare in the case of gynaecological malignoma.

The invention also relates to the use of one or more marker sequences according to the invention, an arrangement according to the invention, a protein array according to the invention, a diagnostic tool according to the invention or a test kit according to the invention for distinguishing gynaecological malignoma from benign changes.

The invention also relates to the use of one or more marker sequences according to the invention, an arrangement according to the invention, a protein array according to the invention, a diagnostic tool according to the invention or a test kit according to the invention for individualised diagnosis and/or therapy in individual patients, patient groups, cohorts, population groups, variants of gynaecological malignoma, or stages of gynaecological malignoma.

The invention also relates to the use of one or more marker sequences according to the invention, an arrangement according to the invention, a protein array according to the invention, a diagnostic tool according to the invention or a test kit according to the invention for the detection and/or for the determination of the quantity of one or more gynaecological malignoma-associated autoantibodies, for example in bodily fluid or tissue of a patient.

The invention also relates to the use of one or more marker sequences according to the invention, an arrangement according to the invention, a protein array according to the invention, a diagnostic tool according to the invention or a test kit according to the invention for the analysis of autoantibody profiles of patients, in particular for the qualitative and/or quantitative analysis of autoantibodies and/or for the monitoring of changes of autoantibody profiles, for example in bodily fluids such as serum, tissue or tissue samples from the patient.

The invention also relates to the use of one or more marker sequences according to the invention, an arrangement according to the invention, a protein array according to the invention, a diagnostic tool according to the invention or a test kit according to the invention for the screening of substances (active agents) for gynaecological malignoma.

The invention also relates to a target for the treatment and/or therapy of gynaecological malignoma, wherein the target is selected from the marker sequences SEQ ID No. 1-1467 according to the invention and partial sequences of SEQ ID No. 1-1467 with at least 90%, preferably 95%, of the length of sequences SEQ ID No. 1-1467, and homologues of SEQ ID No. 1-1467 and partial sequences thereof with an identity of at least 95%, preferably 98% or more, to the corresponding nucleic acid and/or protein sequences.

The invention also relates to a method for the early detection, diagnosis, prognosis, therapy control and/or aftercare in the case of gynaecological malignoma, wherein

a.) a marker sequence or a number of marker sequences selected from the group comprising the sequences SEQ ID No. 1-1467 and partial sequences of SEQ ID No. 1-1467 with at least 90%, preferably 95%, of the length of the sequences SEQ ID No. 1-1467, and homologues of SEQ ID No. 1-1467 and partial sequences thereof with an identity of at least 95%, preferably 98% or more, to the corresponding nucleic acid and/or protein sequences and sequences coded by SEQ ID No. 1-489, partial sequences thereof and homologues thereof is/are applied to a support,
b.) is/are brought into contact with bodily fluid or tissue sample from a patient, and
c.) an interaction of the bodily fluid or of the tissue sample with the marker sequence(s) for gynaecological malignoma from a.) is detected.

The invention relates to the use of one or more marker sequences for gynaecological malignoma for the early detection, diagnosis, prognosis and/or therapy control in the case of gynaecological malignoma, wherein the marker sequence(s) for gynaecological malignoma is/are selected from the sequences SEQ ID No. 1-978 and partial sequences of SEQ ID No. 1-978 with at least 90%, preferably at least 95%, of the length of SEQ ID No. 1-978, and homologues of SEQ ID No. 1-978 and partial sequences thereof with an identity of at least 95%, preferably at least 98% or more, to the corresponding sequences and proteins/peptides coded by the sequences SEQ ID No. 1-978, coded by the partial sequences, and the homologues and sequences coded by SEQ ID No. 1-489, partial sequences thereof and homologues thereof.

The invention relates to the use of one or more marker sequences according to the invention for gynaecological malignoma for the individualised diagnosis and/or therapy in individual patients, patient groups, cohorts, population groups, variants of gynaecological malignoma or stages of gynaecological malignoma.

The invention relates to the use of one or more marker sequences according to the invention for gynaecological malignoma for the detection and/or for the determination of the quantity of one or more gynaecological malignoma-associated autoantibodies, for example in bodily fluid or tissue of a patient.

The invention also relates to marker sequences for gynaecological malignoma selected from the sequences comprising SEQ ID No. 1-489 and partial sequences of SEQ ID No. 1-489 with at least 90%, preferably 95%, of the length of the sequences SEQ ID No. 1-489, and homologues of SEQ ID No. 1-489 and partial sequences thereof with an identity of at least 95%, preferably 98% or more, to the corresponding sequences and proteins/peptides coded by the sequences SEQ ID No. 1-489, coded by partial sequences thereof, and the corresponding homologues of these sequences.

The invention relates to an arrangement of one or more marker sequences for gynaecological malignoma on a support for the early detection, diagnosis, prognosis and/or therapy control in the case of gynaecological malignoma, wherein the marker sequence(s) for gynaecological malignoma is/are selected from groups of proteins SEQ ID No. 979 to 1467 and of proteins coded by sequences SEQ ID No. 1-978 and coded by partial sequences of SEQ ID No. 1-978 with at least 90%, preferably 95% or more, of the length of the sequences SEQ ID No. 1-978 and coded by homologues of SEQ ID No. 1-978 and partial sequences thereof with an identity of at least 95%, preferably 98% or more, to the corresponding sequences.

The invention relates to an arrangement according to the invention, wherein the marker sequence(s) for gynaecological malignoma is/are applied to a solid support, in particular a filter, a membrane, a magnetic or fluorophore-labelled bead, a silicon wafer, glass, metal, plastic, a chip, a mass spectrometry target or a matrix.

The invention relates to an arrangement according to the invention or a use according to the invention of one or more marker sequences for gynaecological malignoma, wherein the marker sequence(s) for gynaecological malignoma is/are present as clone(s).

The invention also relates to an assay or protein biochip comprising an arrangement according to the invention or one or more marker sequences according to the invention for gynaecological malignoma.

The invention also relates to a diagnostic tool (test kit) for the early detection and/or diagnosis of gynaecological malignoma and/or prognosis and/or prediction of the risk of metastasis formation in the case of gynaecological malignoma and/or for therapy monitoring and/or for aftercare in the case of gynaecological malignoma, comprising
a. an arrangement according to the invention or an assay or protein array according to the invention, or one or more marker sequences for gynaecological malignoma selected from the group comprising SEQ ID No. 1-1467 and partial sequences of SEQ ID No. 1-1467 with at least 90%, preferably 95%, of the length of the sequences SEQ ID No. 1-1467, and homologues of SEQ ID No. 1-1467 and partial sequences thereof with an identity of at least 95%, preferably 98% or more, to the corresponding nucleic acid and/or protein sequences and sequences coded by SEQ ID No. 1-489, partial sequences thereof and homologues thereof, and
b. optionally further additives and excipients.

The invention also relates to a method for identifying marker sequences for gynaecological malignoma comprising the following steps
a. providing sequences on an array,
b. identifying marker sequence candidates for gynaecological malignoma by comparative analysis of the signals measured in the event of contact of the marker sequence candidates with bodily fluid or tissue sample from a patient with gynaecological malignoma and
   bodily fluid or tissue sample from a patient without gynaecological malignoma,
c. characterising the marker sequence candidates for gynaecological malignoma with the aid of a protein array,
d. selecting marker sequences for gynaecological malignoma, which are characterised in that they deliver a different signal in the case of patients with gynaecological malignoma and patients without gynaecological malignoma (marker sequence(s) for gynaecological malignoma).

The invention relates to an arrangement of marker sequences for gynaecological malignoma on a support for the early detection, diagnosis, prognosis, and therapy control with one or more different marker sequences for gynaecological malignoma selected from the group of proteins SEQ ID No. 979 to 1467 and/or partial sequences of these proteins and/or coded by sequences SEQ ID No. 1-489 (clone sequences, cDNA) and/or coded by sequences SEQ ID. No. 490-978 (RNA sequences) and/or coded by partial sequences of SEQ ID. No. 1-978.

Within the scope of this invention, a use for the proteins SEQ ID No. 979 to 1467 and/or partial sequences of these proteins and/or proteins coded by sequences SEQ ID No. 1-489 and/or proteins coded by sequences SEQ ID. No. 490-978 and/or coded by partial sequences of SEQ ID. No. 1-978 has been found for the first time for gynaecological malignoma and has been implemented in the arrangement according to the invention. The invention thus provides a panel of marker sequences for gynaecological malignoma that can be used within the scope of individualised diagnosis and therapy in order to diagnose gynaecological malignoma and to monitor the therapy in a targeted and individually adapted manner in different patients, patient groups, cohorts, population groups, variants of gynaecological malignoma, etc.

The invention also relates to the use of a marker sequence of a number of marker sequences for gynaecological malignoma selected from the group comprising SEQ ID No. 1-1467 and partial sequences of SEQ ID No. 1-1467 with at least 90%, preferably 95%, of the length of the sequences SEQ ID No. 1-1467, and homologues of SEQ ID No. 1-1467 and partial sequences thereof with an identity of at least 95%, preferably 98% or more, to the corresponding nucleic acid and/or protein sequences, in particular proteins SEQ ID No. 979 to 1467 and/or partial sequences of these proteins and/or coded by sequences SEQ ID No. 1-489 and/or coded by sequences SEQ ID. No. 490-978 and/or coded by partial sequences of SEQ ID. No. 1-978 for diagnosis and therapy of gynaecological malignoma, in particular for the early detection of gynaecological malignoma, for the diagnosis of gynaecological malignoma, for the prognosis, for example of the risk of metastasis formation, therapy control, for example prediction and monitoring of the response to a drug or a therapy (prediction of the sensitivity or resistance), or aftercare. In particular, the invention also relates to the detection and the determination of the quantity of at least two different autoantibodies in a patient, wherein at least two different marker sequences for gynaecological malignoma according to the invention are used accordingly (as antigens).

In preferred embodiments of the invention, the arrangement/use according to the invention comprises 2 or 3, preferably 4 or 5, particularly preferably 7 or 8 or more, different marker sequences for gynaecological malignoma. The arrangement/use may comprise 9 or 10 or more marker sequences for gynaecological malignoma, for example 10 to 50, preferably 50 to 100 or more. In accordance with the invention, arrangements/uses that are produced for patients individually, for example within the scope of individualised (personalised) medicine, are also included. The invention thus also relates to an arrangement/use, wherein at least 2 to 5 or 10, preferably 30 to 50 marker sequences for gynaecological malignoma or 50 to 100 or more marker sequences for gynaecological malignoma are determined on or relative to a patient to be tested.

A preferred embodiment of the invention concerns an arrangement/use, characterised in that the marker sequences for gynaecological malignoma are applied to a solid support, in particular a filter, a membrane, a bead, for example a magnetic or fluorophore-labelled bead, a silicon wafer, glass, metal, plastic, a chip, a mass spectrometry target or a matrix. However, a filter or a bead is preferred in accordance with the invention.

Furthermore, PVDF, nitrocellulose or nylon is preferred as a filter (for example Immobilon P Millipore, Protran Whatman, Hybond N+ Amersham).

A further embodiment concerns an arrangement/use, characterised in that the marker sequences for gynaecological malignoma are present as clones.

The invention therefore relates to the use of marker sequences for gynaecological malignoma for the diagnosis of gynaecological malignoma, wherein at least one marker sequence for gynaecological malignoma of a DNA, in particular cDNA selected from the group SEQ ID No. 1-489 or RNA selected from the group 490-978 or a partial sequence or a homolog sequence thereof is determined on or relative to a patient to be tested.

The provision of marker sequences for gynaecological malignoma (also referred to as marker sequences according to the invention) allows a reliable diagnosis and stratification of patients with gynaecological malignoma, in particular by means of a protein array (also referred to as a protein biochip).

The marker sequences according to the invention for gynaecological malignoma were able to be identified by means of differential screening of samples, more specifically from healthy test subjects, with patient samples with gynaecological malignoma. Here, these marker sequences according to the invention were able to be identified for the first time by means of protein array (see the examples).

The term “gynaecological malignoma” comprises a group of diseases that can be preliminary stages of gynaecological malignoma and the establishment thereof as gynaecological malignoma. Malignant diseases of the genital tract in women, in particular malignant diseases of the cervix, such as cervical carcinoma, in particular invasive cervical carcinoma (definition for example in accordance with Pachyrembel, de Gruyter, 263^{rd} edition (2012), Berlin), are included. Variants of gynaecological malignoma and stages of gynaecological malignoma are also defined in Pschyrembel.

In a further embodiment of the invention (for example arrangement, use), the marker sequences according to the invention for gynaecological malignoma can also be combined with, supplemented or extended by known biomarkers for this indication. However, at least 50%, preferably 60%, particularly preferably 70% or more, marker sequences according to the invention are represented here, for example in the arrangement according to the invention. In particularly preferred embodiments of the invention, in particular of the arrangement according to the invention, the assay according to the invention and protein array and also the use according to the invention, at least 75%, preferably 80% or 85%, particularly preferably 90% or 95%, of marker sequences according to the invention are represented.

In a preferred embodiment, the marker sequences for gynaecological malignoma are determined outside the human body, and the determination is performed in an ex vivo/in vitro diagnosis.

The invention also relates to an assay or protein array comprising an arrangement/use according to the invention. The invention relates to a diagnostic device and/or an assay, in particular a protein array, that allows an early detection, diagnosis, prognosis, stratification and/or testing for gynaecological malignoma.

The invention also relates to the use of an arrangement according to the invention or of an assay or protein array according to the invention for the analysis of autoantibody profiles of patients, in particular for the quantitative analysis and/or for the monitoring of changes of autoantibody profiles of patients.

The invention also relates to a diagnostic tool (test kit) for the early detection and/or diagnosis of gynaecological malignoma and/or prognosis and/or prediction of the risk of metastasis formation in the case of gynaecological malignoma, for example comprising an arrangement according to the invention, preferably on a support or an assay or protein array according to the invention and optionally further additives and excipients. The invention also relates to a corresponding diagnostic tool (test kit) for therapy monitoring and/or aftercare in the case of gynaecological malignoma.

In a further embodiment of the invention, the invention relates to the use of marker sequences for gynaecological malignoma as diagnostic agents, wherein at least one marker sequence of a cDNA is selected from the group SEQ ID No. 1-489 (clone sequences) or SEQ ID No. 490-978 (RNA) or is a protein coded by SEQ ID No. 1-978 or a partial sequence or fragment thereof.

The invention also relates to a method for the early detection and diagnosis of gynaecological malignoma, wherein

a.) a marker sequence or a number of marker sequences for gynaecological malignoma selected from the group of proteins SEQ ID No. 979 to 1467 and/or partial sequences of these proteins and/or coded by sequences SEQ ID No. 1-489 and/or coded by sequences SEQ ID. No. 490-978 and/or coded by partial sequences of SEQ ID No. 1-978 is/are applied to a support and
b.) is/are brought into contact with bodily fluid or tissue sample from a patient, and
c.) an interaction of the bodily fluid or tissue sample with the marker sequences for gynaecological malignoma from a.) is detected.

The invention also relates to a method for the early detection and diagnosis of gynaecological malignoma, wherein

a.) at least one marker sequence for gynaecological malignoma of a cDNA selected from the group SEQ ID No. 1-489 (clone sequences) or SEQ ID No. 490-978 (RNA) or a partial sequence of SEQ ID. No. 1-978 is applied to a support and
b.) is brought into contact with the bodily fluid or tissue sample from a patient, and
c.) an interaction of the bodily fluid or tissue sample with the marker sequences from a.) is detected.

A particular embodiment of the invention concerns methods for the early detection and diagnosis of gynaecological malignoma, wherein the interaction according to c.) indicates a gynaecological malignoma-associated autoantibody profile of the patient or of a cohort or of a population group or of a specific disease progression (prognosis) or of a certain response to a therapy/drug.

A marker sequence of a number of marker sequences for gynaecological malignoma is/are used in a diagnosis method and/or in a diagnostic tool. In a preferred embodiment, at least 2, for example 3, 4, 5, 6, 7, 8, 9, 10, preferably 15 to 20 marker sequences or 30 to 50 or 100 or more marker sequences for gynaecological malignoma are used together or in combination, for example directly in succession or in parallel.

An interaction of the bodily fluid or of the tissue sample with the marker sequence or marker sequences for gynaecological malignoma can be detected for example by means of a probe, in particular by means of an antibody.

The invention also relates to a method for the stratification, in particular for risk stratification, or for the therapy control of a patient with gynaecological malignoma, wherein the gynaecological malignoma-associated autoantibody profile of a patient is determined and optionally monitored with the aid of one or more marker sequences for gynaecological malignoma selected from the group of proteins SEQ ID No. 979 to 1467 and/or partial sequences of these proteins and/or coded by sequences SEQ ID No. 1-489 and/or coded by sequences SEQ ID. No. 490-978 and/or coded by partial sequences of SEQ ID No. 1-978. A particular embodiment of the invention relates to the method, wherein the stratification or the therapy control comprises decisions for the treatment and therapy of the patient, in particular hospitalisation of the patient, use, efficacy and/or dosage of one or more drugs, a therapeutic measure or the monitoring of the course of a disease and the course of therapy, aetiology or classification of a disease including prognosis.

The invention also relates to a method for the stratification, in particular for the risk stratification or therapy control of a patient with gynaecological malignoma, wherein at least one marker sequence of a nucleic acid, for example cDNA selected from the group SEQ ID No. 1-489 (clone sequences) or SEQ ID No. 490-978 (RNA) or a protein coded thereby or a partial sequence of SEQ ID No. 1-978 is determined on a patient to be tested. The invention also relates to a method for the stratification, in particular for the risk stratification and/or therapy control of a patient with gynaecological malignoma, wherein at least one marker sequence of a DNA, cDNA selected from the group SEQ ID No. 1-489 (clone sequences) or SEQ ID No. 490-978 (RNA or DNA) or a partial sequence thereof is used in order to detect, to identify or to monitor gynaecological malignoma-associated autoantibodies and/or autoantibody profiles in the patient.

Autoantibody profiles comprise the quantity of one or more autoantibodies of which the occurrence/expression accompanies the development and/or establishment of gynaecological malignoma.

The stratification of patients with gynaecological malignoma in new or established sub-groups of patients with gynaecological malignoma, and the appropriate selection of patient groups for the clinical development of new therapeutic agents is also included. The term “therapy control” also includes the allocation of patients to responders and non-responders regarding a therapy or the therapy course thereof.

In the sense of this invention, “diagnosis” means the positive determination of gynaecological malignoma by means of the marker sequences for gynaecological malignoma according to the invention as well as the assignment of the patients to gynaecological malignoma. The term diagnosis includes the medical diagnostics and examinations in this regard, in particular in-vitro diagnostics and laboratory diagnostics, and also proteomics and nucleic acid blotting. Further tests may be necessary to be sure and to exclude other diseases. The term diagnosis therefore also includes the differential diagnosis of gynaecological malignoma by means of the marker sequences for gynaecological malignoma according to the invention, and the prognosis of gynaecological malignoma, in particular the prediction of the risk of metastasis formation.

In the sense of this invention, “stratification or therapy control” means that, for example, the methods according to the invention allow decisions for the treatment and therapy of the patient, whether it is the hospitalisation of the patient, the use, efficacy and/or dosage of one or more drugs, a therapeutic measure or the monitoring of the course of a disease and the course of therapy or aetiology or classification of a disease, for example into a new or existing sub-type, or the differentiation of diseases and patients thereof.

In a further embodiment of the invention, the term “stratification” in particular includes the risk stratification with the prognosis of an “outcome” of a negative health event.

“Prognosis” means the prediction of the course of a disease, for example the prediction of the relapse-free survival, the overall survival, or the risk of metastasis formation.

Within the scope of this invention, the term “patient” is understood to mean any test subject (human or mammal), with the provision that the test subject is tested for gynaecological malignoma. The term “female patient” is understood to mean any female test subject. The terms “healthy subject” or “control” or “healthy control individual” are understood to mean any test subject, preferably a female test subject, who does not have gynaecological malignoma or any benign change or in whom this cannot be detected with the known standard methods (for example see Pschyrembl, above).

The term “marker sequence for gynaecological malignoma” in the context of this invention means that that the nucleic acid, for example DNA, in particular cDNA or RNA or the amino acid sequence or the polypeptide or protein obtainable therefrom or the amino acids (protein, peptide) coded by the nucleic acids are significant (specific) for gynaecological malignoma. Marker sequences for gynaecological malignoma can be nucleic acid sequences and amino acid sequences, wherein modifications are also included.

The expression “for gynaecological malignoma” means that, for example, the cDNA or the polypeptide or protein obtainable therefrom interacts with substances from the bodily fluid or tissue sample from a patient with gynaecological malignoma (for example antigen (epitope)/antibody (paratope) interaction). These substances from the bodily fluid or tissue sample either only occur or are only expressed, or occur or are expressed at least in an intensified manner, in the case of gynaecological malignoma, whereas these substances in patients or individuals without gynaecological malignoma are not present or are only present to a smaller extent (smaller quantity, lower concentration). On the other hand, marker sequences for gynaecological malignoma can also be characterised in that they interact with substances from the bodily fluid or tissue sample from patients with gynaecological malignoma because these substances no longer occur or are no longer expressed, or occur or are expressed at least in a much lower quantity/concentration, in the case of gynaecological malignoma, whereas these substances are present to a much greater extent in patients or individuals without gynaecological malignoma. Marker sequences for gynaecological malignoma may also be present in healthy test subjects, however the quantity (concentration) thereof changes for example with the development, establishment and therapy of gynaecological malignoma. The marker sequences for gynaecological malignoma are therefore biomarkers for gynaecological malignoma. The marker sequences for gynaecological malignoma may thus indicate a profile of substances from bodily fluid and tissue sampling, for example an autoantibody profile for gynaecological malignoma.

“Autoantibody profile for gynaecological malignoma” thus includes on the one hand the composition (one or more autoantibodies) and on the other the quantity/concentration of individual autoantibodies. Here, the composition and/or the quantity or concentration are specific for gynaecological malignoma.

In a particularly preferred embodiment of the invention, the marker sequence for gynaecological malignoma is an antigen or part of an antigen or codes for an antigen or for part of an antigen.

In a particularly preferred embodiment, the marker sequence for gynaecological malignoma identifies/binds to autoantibodies that are present (intensified) during the course of the development, establishment and therapy of gynaecological malignoma or are present to a smaller extent (or are no longer present) (referred to hereinafter as “autoantibodies for gynaecological malignoma”). Autoantibodies are formed by the body against the body's own antigens, which for example are produced when gynaecological malignoma is present. Autoantibodies are formed by the body against different substances and pathogens. Within the scope of the present invention, the autoantibodies for gynaecological malignoma in particular that are formed with the occurrence of and during the course of the development of gynaecological malignoma and/or of which the expression is upregulated or downregulated are detected. Gynaecological malignoma-associated autoantibodies can be detected with the aid of the method according to the invention and marker sequences for gynaecological malignoma and are therefore used as an indication for gynaecological malignoma. The detection and the monitoring of the quantity of autoantibodies for gynaecological malignoma in the patient can be used for the early detection, diagnosis and/or therapy monitoring/therapy control. These gynaecological malignoma-associated autoantibody profiles may be sufficiently characterised already with use of a marker sequence for gynaecological malignoma. In other cases, two or more marker sequences for gynaecological malignoma are necessary in order to indicate a gynaecological malignoma-associated autoantibody profile.

In preferred embodiments of the invention, the gynaecological malignoma-associated autoantibodies can be detected using marker sequences for gynaecological malignoma, which are derived from another individual, because they originate for example from a commercial cDNA bank or can be compared with a gold standard. In other preferred embodiments of the invention, the autoantibodies for gynaecological malignoma can be detected using marker sequences for gynaecological malignoma, which are derived from the same individual (autoantigen), because they originate for example from a cDNA bank produced especially for the patient or a group of patients (for example within the scope of personalised medicine).

Autoantibodies can be formed by the patient already many years before the occurrence of the first symptoms of the disease. Early detection, diagnosis and also prognosis and (preventative) treatment would therefore be possible years before the visible outbreak of the disease. The devices and means (arrangement, array, protein biochip, diagnostic tool, test kit) and methods according to the invention thus enable a very early intervention compared with known methods, which considerably improves the prognosis and survival rates. Since the gynaecological malignoma-associated autoantibody profiles change during the establishment and treatment/therapy of gynaecological malignoma, the invention also enables the detection and the monitoring of gynaecological malignoma at any stage of development and treatment and also monitoring within the scope of aftercare in the case of gynaecological malignoma. The means according to the invention also allow easy handling, for example at home, by the patient themself and cost-effective routine precautionary measures for early detection and also aftercare.

In particular due to the use of antigens as specific marker sequence for gynaecological malignoma, which derive from sequences already known, for example from commercial cDNA banks, test subjects (individuals) can be tested, and, where applicable, gynaecological malignoma-associated autoantibodies present in these test subjects can be detected, even if the corresponding autoantigens are not (yet) known in these test subjects.

Different patients may have different gynaecological malignoma-associated autoantibody profiles, for example different cohorts or population groups differ from one another. Here, each patient may form one or more different gynaecological malignoma-associated autoantibodies during the course of the development of gynaecological malignoma and the progression of the disease of gynaecological malignoma, that is to say also different autoantibody profiles. In addition, the composition and/or the quantity of the formed gynaecological malignoma-associated autoantibodies may change during the course of the gynaecological malignoma development and progression of the disease, such that a quantitative evaluation is necessary. The therapy/treatment of gynaecological malignoma also leads to changes in the composition and/or the quantity of gynaecological malignoma-associated autoantibodies. The large selection of marker sequences for gynaecological malignoma according to the invention allows the individual compilation of marker sequences for gynaecological malignoma in an arrangement for individual patients, groups of patients, certain cohorts, population groups, etc. In an individual case, the use of a marker sequence for gynaecological malignoma may therefore be sufficient, whereas in other cases at least two or more marker sequences for gynaecological malignoma have to be used together or in combination in order to produce a meaningful autoantibody profile.

Compared with other biomarkers, the detection of gynaecological malignoma-associated autoantibodies for example in the serum/plasma has the advantage of high stability and storage capability and good detectability. The presence of autoantibodies also is not subject to a circadian rhythm, and therefore the sampling is independent of the time of day, food intake and the like.

In addition, gynaecological malignoma-associated autoantibodies can be detected with the aid of the corresponding antigens/autoantigens in known assays, such as ELISA or Western Blot, and the results can be checked for this.

In the sense of the invention, “wherein at least one marker sequence for gynaecological malignoma is selected” means that an interaction is detected. Such an interaction is, for example, a bond, in particular a binding substance on at least one marker sequence for gynaecological malignoma, or, in the case that the marker sequence for gynaecological malignoma is a nucleic acid, for example a cDNA, the hybridisation with a suitable substance under selected conditions, in particular stringent conditions (for example as defined conventionally in J. Sambrook, B. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology”, Green Publishing Associates and Wiley Interscience, N.Y. (1989)). One example of stringent hybridisation conditions is: hybridisation in 4×SSC at 65° C. (alternatively in 50% formamide and 4×SSC at 42° C.), followed by a number of washing steps in 0.1×SSC at 65° C. for a total of approximately one hour. An example of less stringent hybridisation conditions is hybridisation in 4×SSC at 37° C., followed by a number of washing steps in 1×SSC at room temperature.

The interaction between the bodily fluid or tissue sample from a patient and the marker sequences for gynaecological malignoma is preferably a protein-protein interaction.

In accordance with the invention, such substances, for example gynaecological malignoma-associated antigens, autoantigens, autoantibodies, are part of a bodily fluid, in particular blood, whole blood, blood plasma, blood serum, patient serum, urine, cerebrospinal fluid, synovial fluid or a tissue sample, for example from tumour tissue from the patient. The invention in particular relates to the use of these bodily fluids and tissue samples for early detection, diagnosis, prognosis, therapy control and aftercare.

However, in a further embodiment of the invention, the marker sequences for gynaecological malignoma or the substances identified from these marker sequences, for example gynaecological malignoma-associated autoantibodies, can be present in a significantly higher or lower expression rate or concentration, which is indicative of gynaecological malignoma. Here, the relative expression rates diseased/healthy of the marker sequences according to the invention for gynaecological malignoma or the substances identified from these marker sequences are determined by means of proteomics or nucleic acid blots.

The marker sequences for gynaecological malignoma, in a further embodiment of the invention, have a recognition signal that is addressed to the substance to be bound (for example antibody, nucleic acid). In accordance with the invention, the recognition signal for a protein is preferably an epitope and/or paratope and/or hapten, and for a cDNA is preferably a hybridisation or binding region.

The marker sequences for gynaecological malignoma according to the invention are detailed in Table A (RNA) and in the sequence protocol and can also be clearly identified by the respectively cited database entry (also accessible by Internet: http://www.ncbi.nlm.nih.gov/) (by means of accession no.).

The invention therefore also concerns the full-length sequences of the marker sequences for gynaecological malignoma according to the invention, more specifically as defined via the known database entry according to Table A and in the sequence protocol, referred to hereinafter as SEQ 1-1467.

Furthermore, analogue embodiments of SEQ 1-1467 to the marker sequences for gynaecological malignoma SEQ 1-1467, for example as presented in the claims, are therefore also included, since the SEQ 1-1467 according to the invention in turn constitute partial sequences, at least with high homology. However, the marker sequences for gynaecological malignoma SEQ 1-1467 are preferred in accordance with the invention.

In accordance with the invention, the marker sequences also comprise modifications of the nucleic acid sequence, in particular cDNA sequence and the corresponding amino acid sequence, such as chemical modification, such as citrullination, acetylation, phosphorylation, glycosylation or polyA strand and further modifications known as appropriate to a person skilled in the art.

The invention also relates to homologues of the marker sequences for gynaecological malignoma and homologues of the partial sequences, for example fragments of marker sequences for gynaecological malignoma. For example, homologues are nucleic acid sequences and/or protein sequences, in particular homologues of SEQ ID No. 1-1467 that have an identity with the marker sequences for gynaecological malignoma of at least 70% or 80%, preferably 90% or 95%, particularly preferably 96% or 97% or more, for example 98% or 99%. In a particularly preferred embodiment of the invention, for the case in which the marker sequences for gynaecological malignoma are antigens, the homology in the sequence range in which the antigen-antibody or antigen-autoantibody interaction takes place, is at least 95%, preferably at least 97%, particularly preferably at least 99%.

The invention also relates to partial sequences of the marker sequences for gynaecological malignoma. Partial sequences also include fragments of the marker sequences according to the invention, and partial sequences are nucleic acids or proteins/peptides that are shortened compared with the entire nucleic acid or the entire protein/peptide. Here, the deletion may occur at the end or the ends and/or within the sequence. For example, partial sequences and/or fragments that have 50 to 100 nucleotides or 70-120 nucleotides of an entire sequence are included, for example of SEQ 1-1467. Homologues of partial sequences and fragments are also included in accordance with the invention. In a particular embodiment, the marker sequences for gynaecological malignoma are shortened compared with the sequences 1-1467 to such an extent that they still consist only of the binding point(s) for the gynaecological malignoma-associated autoantibody in question. In accordance with the invention, marker sequences for gynaecological malignoma are also included that differ from the sequences SEQ ID No. 1-1467 in that they contain one or more insertions, wherein the insertions for example are 1 to 100 or more nucleotide/amino acids long, preferably 5 to 50, particularly preferably 10 to 20 nucleotides/amino acids long and the sequences are otherwise identical however or homologous to sequences 1-1467. In accordance with the invention, partial sequences that have at least 90%, preferably at least 95%, of the length of the sequences in question are preferred.

In a further embodiment, the respective marker sequence for gynaecological malignoma can be represented in different quantities in one or more regions on the support. This allows a variation of the sensitivity. The regions may each have a totality of marker sequences for gynaecological malignoma, that is to say a sufficient number of different marker sequences for gynaecological malignoma, in particular 2, 3, 4, 5, 6, 7, 8, 9 or 10 or more different and where applicable further nucleic acids and/or proteins, in particular biomarkers.

In a particularly preferred embodiment of the invention, at least 96 to 25,000 (numerically) or more different or same marker sequences for gynaecological malignoma and optionally further nucleic acids and/or proteins, in particular biomarkers, are represented on the support. Further preferably, more than 2,500, particularly preferably 10,000 or more, different or same marker sequences for gynaecological malignoma and optionally further nucleic acids and/or proteins, in particular biomarkers, are represented on the support.

Within the scope of this invention, “arrangement” is synonymous with “array”, and, if this “array” is used to identify substances on marker sequences for gynaecological malignoma, this is to be understood preferably to be an “assay” or a diagnostic device. In a preferred embodiment, the arrangement is designed such that the marker sequences for gynaecological malignoma represented on the arrangement are present in the form of a grid on a support. Furthermore, those arrangements are preferred that permit a high-density arrangement of marker sequences for gynaecological malignoma, for example protein binders. The marker sequences for gynaecological malignoma are preferably spotted. Such high-density spotted arrangements are disclosed for example in WO 99/57311 and WO 99/57312 and can be used advantageously in a robot-supported automated high-throughput method.

Within the scope of this invention, however, the term “assay” or diagnostic device likewise comprises those embodiments of a device such as ELISA, bead-based assay, line assay, Western Blot, and immunochromatographic methods (for example what are known as lateral flow immunoassays) or similar immunological single or multiplex detection methods.

A “protein array” (also referred to as a protein biochip) in the sense of this invention is the systematic arrangement of marker sequences for gynaecological malignoma on a solid support, wherein the marker sequences for gynaecological malignoma are proteins or peptides or parts thereof.

The marker sequences for gynaecological malignoma of the arrangement are fixed on a solid support, but are preferably spotted or immobilised or even printed on, that is to say applied in a reproducible manner. One or more marker sequences for gynaecological malignoma can be present multiple times in the totality of all marker sequences for gynaecological malignoma and may be present in different quantities based on a spot. Furthermore, the marker sequences for gynaecological malignoma can be standardised on the solid support (for example by means of serial dilution series of, for example, human globulins as internal calibrators for data normalisation and quantitative evaluation).

In a further embodiment, the marker sequences for gynaecological malignoma are present as clones. Such clones can be obtained for example by means of a cDNA expression library according to the invention (Bussow et al. 1998 (above)). In a preferred embodiment, such expression libraries containing clones are obtained using expression vectors from a cDNA expression library consisting of the cDNA marker sequences. These expression vectors preferably contain inducible promoters. The induction of the expression can be carried out for example by means of an inducer, such as IPTG. Suitable expression vectors are described in Terpe et al. (Terpe T Appl Microbiol Biotechnol. 2003 January; 60(5):523-33).

Expression libraries are known to a person skilled in the art; they can be produced in accordance with standard works, such as Sambrook et al, “Molecular Cloning, A laboratory handbook, 2nd edition (1989), CSH press, Cold Spring Harbor, N.Y. Expression libraries that are tissue-specific (for example human tissue, in particular human organs, for example from tissue from the ovaries and cervix) are furthermore preferable. Further, expression libraries that can be obtained by means of exon-trapping are also included in accordance with the invention. Instead of the term expression library, reference may also be made synonymously to an expression bank.

Protein arrays or corresponding expression libraries that do not exhibit any redundancy (what is known as a Uniclone® library) and that can be produced for example in accordance with the teaching of WO 99/57311 and WO 99/57312 are furthermore preferred. These preferred Uniclone® libraries have a high proportion of non-defective fully expressed proteins of a cDNA expression library.

Within the scope of this invention, the clones can also be, but are not limited to, transformed bacteria, recombinant phages or transformed cells of mammals, insects, fungi, yeasts or plants.

The clones are fixed, spotted or immobilised on a solid support. The invention therefore relates to an arrangement/use, wherein the marker sequences for gynaecological malignoma are present as clones.

In addition, the marker sequences for gynaecological malignoma can be present in the respective form in the form of a fusion protein, which for example contains at least one affinity epitope or “tag”. The tag may be or may contain one such as c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

In a further preferred embodiment of the arrangement/use according to the invention, this corresponds to a grid with the dimensions of a microtiter plate (8-12 well strips, 96 wells, 384 wells or more), a silicon wafer, a chip, a mass spectrometry target or a matrix.

In a further particularly preferred embodiment of the invention, the arrangement is located on a bead or a small plate.

In a further embodiment, the invention relates to an assay or protein array for identifying and characterising a substance (for example also referred to as a hit, lead substance, candidate, active agent) for gynaecological malignoma, characterised in that an arrangement or assay according to the invention

a.) is brought into contact with at least one substance to be tested, and
b.) binding success is detected.

The substance to be tested may be any native or non-native biomolecule, a (synthetic) chemical molecule, a natural substance, a mixture or a substance library.

Once the substance to be tested has contacted a marker sequence for gynaecological malignoma, the binding success is evaluated, and is performed for example with use of commercially available image analysing software (GenePix Pro (Axon Laboratories), Aida (Raytest), ScanArray (Packard Bioscience).

Binding according to the invention, binding success, interactions, for example protein-protein interactions (for example protein to marker sequence for gynaecological malignoma, such as antigen/antibody) or corresponding “means for detecting the binding success” can be visualised for example by means of fluorescence labelling, biotinylation, radio-isotope labelling or colloid gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemoluminescent substrates. A readout is performed for example by means of a microarray laser scanner, a CCD camera or visually.

In a further embodiment, the invention relates to a drug/active agent or prodrug for gynaecological malignoma developed and obtainable by the use of a marker sequence according to the invention, an arrangement according to the invention, a use according to the invention, or an assay according to the invention.

The invention also relates to the use of a marker sequence for gynaecological malignoma selected from the group of proteins SEQ ID No. 979 to 1467, partial sequences of these proteins and the sequences coding for these proteins, sequences SEQ ID No. 1-489 and SEQ ID. No. 490-978 and partial sequences of SEQ ID No 1-978 and also of proteins coded by SEQ ID No. 1-978 as affinity material for carrying out an apheresis or blood washing for patients with gynaecological malignoma. The invention thus relates to the use of the marker sequences according to the invention, preferably in the form of an arrangement, as affinity material for carrying out a blood washing in the broader sense, wherein substances from bodily fluids from a patient with gynaecological malignoma, such as blood or plasma, bind to the marker sequences according to the invention and consequently can be removed selectively from the bodily fluid.

The following examples explain the invention, but do not limit the invention to the examples.

The examples were carried out with use of the UNIarray technology platform on the basis of quantitative analyses of the autoantibody profiles in the serum of female patients with gynaecological malignoma. Gynaecological malignoma-associated antigens and gynaecological malignoma-associated autoantigens (“biomarkers”), which enable an early detection of gynaecological malignoma and/or indicate a specific form of progression (prognostic relevance), are thus to be identified systematically.

### EXAMPLE 1

Candidates for marker sequences for gynaecological malignoma were identified first.

In the first phase, 50 serum samples are tested for this purpose from female patients with gynaecological malignoma on a MACROarray (comprises approximately 10,000 different recombinant human proteins). Here, candidates for marker sequences for gynaecological malignoma are identified.

### EXAMPLE 2

In the subsequent test phase, these candidates for marker sequences for gynaecological malignoma are analysed comparatively on serum samples from 100 female patients with gynaecological malignoma and 100 female patients with benign changes or 100 healthy control female patients and characterised. As a result of this comparative analysis, marker sequences are primarily identified that interact with gynaecological malignoma-associated autoantibodies.

### EXAMPLE 3

Particularly significant biomarkers (marker sequences for gynaecological malignoma) are selected by means of bioinformatic analysis. The candidates for marker sequences for gynaecological malignoma are evaluated in terms of whether they discriminate between different test subjects (for example healthy/unhealthy)/patient groups (for example low/high risk of metastasis formation)/cohorts (for example certain past histories).

To this end, the marker sequence candidates are applied to a biochip and validated. The data evaluation is performed via statistical analyses, for example threshold value analysis, support vector machine algorithm (SVM). The sample consumption for the validation is just 50 μl/sample. In a first approach, cohorts of category I and II are selected in this way.

The biochip obtained is specific for gynaecological malignoma. This biochip comprises one or more marker sequences for gynaecological malignoma and identifies gynaecological malignoma-associated autoantibodies.

Cohort I: clinical finding: gynaecological malignoma-positive group (CASE group; verified via histopathological finding of the biopsy).

Cohort II: clinical finding: gynaecological malignoma-negative group (control group), age-matched.

Female patients are selected in accordance with inclusion and exclusion criteria

Inclusion criteria
histologically verified gynaecological malignoma
histologically verified non-neoplastic change
healthy age-matched control
at the moment of diagnosis (prior to operation)
prior to or during neoadjuvant and adjuvant anti-oestrogen therapy, adjuvant chemotherapy or adjuvant aromatase inhibitor therapy.

Exclusion criteria
age below 18 years
tumour treatment already administered

Corresponding biochips are developed for diagnosis, prediction of the course of therapy and prediction of metastasis formation.

### EXAMPLE 4

For the development of a protein biochip for the diagnosis of gynaecological malignoma, the results of the autoantibody analysis are compared with the golden standard of diagnosis and the identified marker sequences are validated (marker sequences for gynaecological malignoma). The results are then correlated with other clinical characteristics of gynaecological malignoma, for example tumour size and malignancy.

### EXAMPLE 5

With the development of a protein biochip for prediction of the course of therapy, a certain autoantibody profile or a certain signal of the protein biochip is correlated with the response of the gynaecological malignoma to a certain therapy. In addition, changes of the autoantibody profile are validated, even with regard to different treatment options (continuous time modelling).

### EXAMPLE 6

With the development of a protein biochip for the prediction of metastasis formation, marker sequences for gynaecological malignoma are selected that interact with autoantibodies for gynaecological malignoma that are suitable as indicators for metastasis formation. Due to the comparison of autoantibodies at the moment of diagnosis of female patients with and without metastasis formation, female patients who have a high metastasis risk can be identified.

Within the scope of the identification and validation of marker sequences for gynaecological malignoma, bioinformatic analyses can be performed. For each serum, reactivities against approximately 2,000 different antigens can be measured for this purpose by means of microarray. This data is used for a ranking of the spotted antigens with respect to their differentiation capability between healthy and diseased sera. This evaluation can be performed by means of the non-parametric Mann-Whitney test on normalised intensity data. For normalisation, an internal standard is used that is also spotted on each chip. Since a p-value is calculated for each antigen, methods for correction of multiple testing are used. As a very conservative approach, a Bonferroni correction is performed and in addition the less restrictive False Discovery Rate (FDR) in accordance with Benjamini & Hochberg is calculated.

Furthermore, the data is used for classification of the sera. Here, different multi-variant methods are used. These are methods from the statistical learning methods, such as Support Vector Machines (SVM), neuronal networks or classification trees, and a threshold value method, which is suitable both for classification and for visual representation of the data.

To avoid overfitting, a 10× cross-validation of the data is performed by way of example.

The sequences according to the invention are specified in the accompanying sequence protocol. (The clone sequences (cDNA) SEQ ID No. 1-489, the RNA sequences SEQ ID. No. 490-978 and the protein sequences SEQ ID No. 979-1467).

TABLE A
Data concerning marker sequences for gynaecological malignoma
(RNA) SEQ ID No. 490-978.

| SEQ | | | |
|---|---|---|---|
| ID | Accession | Alias RNA | |
| No. | No. | Accession | Blast |
| 490 | gi\|122114640 | NM_032924.3 | zinc finger protein 3 isoform |
| | | | 2 [Homo sapiens] |
| 491 | gi\|27881505 | NM_007189.1 | ATP-binding cassette sub- |
| | | | family F member 2 isoform a |
| | | | [Homo sapiens] |
| 492 | gi\|194306637 | NM_144985.3 | cadherin-24 isoform 2 [Homo |
| | | | sapiens] |
| 493 | gi\|261337182 | NM_182905.4 | WAS protein family homolog 1 |
| | | | [Homo sapiens] |
| 494 | gi\|16877437 | BC016965.1 | NLRP1 protein [Homo sapiens] |
| 495 | gi\|16332363 | NM_033489.1 | cyclin-dependent kinase 11B |
| | | | isoform 5 [Homo sapiens] |
| 496 | gi\|24797087 | NM_002617.3 | peroxisome biogenesis factor |
| | | | 10 isoform 2 [Homo sapiens] |
| 497 | gi\|51477699 | NM_001003891.1 | mediator of RNA polymerase II |
| | | | transcription subunit 15 |
| | | | isoform a [Homo sapiens] |
| 498 | gi\|215272336 | NM_018667.3 | sphingomyelin |
| | | | phosphodiesterase 3 [Homo |
| | | | sapiens] |
| 499 | gi\|39644878 | BC009967.2 | SCYL1 protein [Homo sapiens] |
| 500 | gi\|156071525 | NM_133171.3 | engulfment and cell motility |
| | | | protein 2 [Homo sapiens] |
| 501 | gi\|145553975 | NM_024657.3 | MORC family CW-type zinc |
| | | | finger protein 4 isoform a |
| | | | [Homo sapiens] |
| 502 | gi\|45387948 | NM_133368.1 | RING finger and SPRY domain- |
| | | | containing protein 1 |
| | | | precursor [Homo sapiens] |
| 503 | gi\|44681483 | NM_152562.2 | cell division cycle- |
| | | | associated protein 2 [Homo |
| | | | sapiens] |
| 504 | gi\|169216540 | XM_001717193.1 | PREDICTED: hypothetical |
| | | | protein [Homo sapiens] |
| 505 | gi\|19584557 | AL713796.1 | hypothetical protein [Homo |
| | | | sapiens] |
| 506 | gi\|55925573 | NM_203404.1 | AMP deaminase 2 isoform 3 |
| | | | [Homo sapiens] |
| 507 | gi\|7020062 | AK000158.1 | unnamed protein product [Homo |
| | | | sapiens] |
| 508 | gi\|41352717 | NM_004416.2 | protein deltex-1 [Homo |
| | | | sapiens] |
| 509 | gi\|190014575 | NM_002775.4 | serine protease HTRA1 |
| | | | precursor [Homo sapiens] |
| 510 | gi\|42544228 | NM_021138.3 | TNF receptor-associated |
| | | | factor 2 [Homo sapiens] |
| 511 | gi\|171543867 | NM_000375.2 | uroporphyrinogen-III synthase |
| | | | [Homo sapiens] |
| 512 | gi\|24307874 | NM_006955.1 | zinc finger protein 33B [Homo |
| | | | sapiens] |
| 513 | gi\|42476323 | NM_003627.4 | large neutral amino acids |
| | | | transporter small subunit 3 |
| | | | [Homo sapiens] |
| 514 | gi\|156071506 | NM_014245.3 | RING-box protein 2 isoform 1 |
| | | | [Homo sapiens] |
| 515 | gi\|44680137 | NM_152414.3 | class E basic helix-loop- |
| | | | helix protein 22 [Homo |
| | | | sapiens] |
| 516 | gi\|154759282 | NM_014603.2 | cerebellar degeneration- |
| | | | related protein 2-like [Homo |
| | | | sapiens] |
| 517 | gi\|156415993 | NM_016037.3 | probable U3 small nucleolar |
| | | | RNA-associated protein 11 |
| | | | [Homo sapiens] |
| 518 | gi\|282847377 | NM_080592.3 | apoptosis-related protein 3 |
| | | | isoform b [Homo sapiens] |
| 519 | gi\|216547614 | NM_019046.2 | ankyrin repeat domain- |
| | | | containing protein 16 isoform |
| | | | a [Homo sapiens] |
| 520 | gi\|54607103 | NM_020187.2 | chromosome 3 open reading |
| | | | frame 37 [Homo sapiens] |
| 521 | gi\|57013271 | NM_001008860.1 | magnesium transporter NIPA2 |
| | | | isoform a [Homo sapiens] |
| 522 | gi\|55770871 | NM_001007102.1 | lethal(3)malignant brain |
| | | | tumor-like protein 3 isoform |
| | | | b [Homo sapiens] |
| 523 | gi\|215599550 | NM_033201.2 | hypothetical protein LOC89927 |
| | | | isoform 1 [Homo sapiens] |
| 524 | gi\|37537683 | NM_007139.2 | zinc finger protein 92 |
| | | | isoform 1 [Homo sapiens] |
| 525 | gi\|116014343 | NM_001624.2 | absent in melanoma 1 protein |
| | | | [Homo sapiens] |
| 526 | gi\|53832030 | NM_004489.4 | G protein pathway suppressor |
| | | | 2 [Homo sapiens] |
| 527 | gi\|18765732 | NM_003081.2 | synaptosomal-associated |
| | | | protein 25 isoform SNAP25A |
| | | | [Homo sapiens] |
| 528 | gi\|195976813 | NM_007117.2 | prothyroliberin precursor |
| | | | [Homo sapiens] |
| 529 | gi\|164519060 | NM_014690.4 | hypothetical protein LOC9715 |
| | | | isoform b [Homo sapiens] |
| 530 | gi\|291327486 | NM_201569.2 | protein SMG7 isoform 4 [Homo |
| | | | sapiens] |
| 531 | gi\|40787998 | NM_012446.2 | single-stranded DNA-binding |
| | | | protein 2 [Homo sapiens] |
| 532 | gi\|28416939 | NM_016038.2 | ribosome maturation protein |
| | | | SBDS [Homo sapiens] |
| 533 | gi\|188528682 | NM_021216.4 | endothelial zinc finger |
| | | | protein induced by tumor |
| | | | necrosis factor alpha [Homo |
| | | | sapiens] |
| 534 | gi\|222080063 | NM_032478.3 | 39S ribosomal protein L38, |
| | | | mitochondrial precursor [Homo |
| | | | sapiens] |
| 535 | gi\|186910285 | NM_024570.2 | ribonuclease H2 subunit B |
| | | | isoform 1 [Homo sapiens] |
| 536 | gi\|87298934 | NM_024712.3 | engulfment and cell motility |
| | | | protein 3 [Homo sapiens] |
| 537 | gi\|300068963 | NM_031207.4 | putative hydroxypyruvate |
| | | | isomerase isoform 1 [Homo |
| | | | sapiens] |
| 538 | gi\|226437572 | NM_032360.3 | acyl-CoA-binding domain- |
| | | | containing protein 6 [Homo |
| | | | sapiens] |
| 539 | gi\|119943095 | NM_032364.5 | dnaJ homolog subfamily C |
| | | | member 14 [Homo sapiens] |
| 540 | gi\|333805595 | NM_152652.2 | zinc finger protein 48 |
| | | | isoform 1 [Homo sapiens] |
| 541 | gi\|18375500 | NM_001641.2 | DNA-(apurinic or apyrimidinic |
| | | | site) lyase [Homo sapiens] |
| 542 | gi\|214010180 | NM_001142276.1 | amyloid-like protein 2 |
| | | | isoform 2 [Homo sapiens] |
| 543 | gi\|104487001 | NM_001040712.1 | receptor-type tyrosine- |
| | | | protein phosphatase delta |
| | | | isoform 5 precursor [Homo |
| | | | sapiens] |
| 544 | gi\|32528305 | NM_002913.3 | replication factor C subunit |
| | | | 1 isoform 1 [Homo sapiens] |
| 545 | gi\|41281488 | NM_014761.2 | IST1 homolog [Homo sapiens] |
| 546 | gi\|7023276 | AK001786.1 | unnamed protein product [Homo |
| | | | sapiens] |
| 547 | gi\|217330575 | NM_007225.2 | neurexophilin-3 precursor |
| | | | [Homo sapiens] |
| 548 | gi\|163310748 | NM_001112720.1 | LIM and calponin homology |
| | | | domains-containing protein 1 |
| | | | isoform e [Homo sapiens] |
| 549 | gi\|221219021 | NM_015157.2 | pleckstrin homology-like |
| | | | domain family B member 1 |
| | | | isoform a [Homo sapiens] |
| 550 | gi\|150456423 | NM_015348.1 | transmembrane protein 131 |
| | | | [Homo sapiens] |
| 551 | gi\|62339397 | NM_014604.2 | tax1-binding protein 3 |
| | | | isoform 1 [Homo sapiens] |
| 552 | gi\|66393092 | AY995135.1 | phospholipase C epsilon 1 |
| | | | [Homo sapiens] |
| 553 | gi\|229576807 | NM_020959.2 | anoctamin-8 [Homo sapiens] |
| 554 | gi\|10863994 | NM_021188.1 | zinc finger protein 410 |
| | | | isoform b [Homo sapiens] |
| 555 | gi\|205277444 | NM_021805.2 | single Ig IL-1-related |
| | | | receptor [Homo sapiens] |
| 556 | gi\|205360980 | NM_024557.3 | protein RIC-3 isoform a [Homo |
| | | | sapiens] |
| 557 | gi\|194018548 | NM_030629.2 | C-Maf-inducing protein |
| | | | isoform Tc-mip [Homo sapiens] |
| 558 | gi\|339895851 | NM_001626.4 | RAC-beta serine/threonine- |
| | | | protein kinase isoform 1 |
| | | | [Homo sapiens] |
| 559 | gi\|148613860 | NM_015548.3 | bullous pemphigoid antigen 1 |
| | | | isoform leA precursor [Homo |
| | | | sapiens] |
| 560 | gi\|4826685 | NM_004939.1 | ATP-dependent RNA helicase |
| | | | DDX1 [Homo sapiens] |
| 561 | gi\|196115280 | NM_002055.3 | glial fibrillary acidic |
| | | | protein isoform 1 [Homo |
| | | | sapiens] |
| 562 | gi\|156104890 | NM_002096.2 | general transcription factor |
| | | | IIF subunit 1 [Homo sapiens] |
| 563 | gi\|167466172 | NM_005346.4 | heat shock 70 kDa protein |
| | | | 1A/1B [Homo sapiens] |
| 564 | gi\|212276187 | NM_002893.3 | histone-binding protein RBBP7 |
| | | | isoform 2 [Homo sapiens] |
| 565 | gi\|88853067 | NM_002911.3 | regulator of nonsense |
| | | | transcripts 1 [Homo sapiens] |
| 566 | gi\|218083729 | NM_001142684.1 | zinc finger MYM-type protein |
| | | | 5 isoform 3 [Homo sapiens] |
| 567 | gi\|219879804 | NM_006700.2 | TRAF-type zinc finger domain- |
| | | | containing protein 1 [Homo |
| | | | sapiens] |
| 568 | gi\|18379335 | NM_006711.2 | RNA-binding protein with |
| | | | serine-rich domain 1 [Homo |
| | | | sapiens] |
| 569 | gi\|68161542 | NM_007074.2 | coronin-1A [Homo sapiens] |
| 570 | gi\|110227862 | NM_016097.3 | immediate early response 3- |
| | | | interacting protein 1 [Homo |
| | | | sapiens] |
| 571 | gi\|253970487 | NM_018677.3 | acetyl-coenzyme A synthetase, |
| | | | cytoplasmic isoform 1 [Homo |
| | | | sapiens] |
| 572 | gi\|52353964 | NM_020170.3 | nicalin precursor [Homo |
| | | | sapiens] |
| 573 | gi\|116174741 | NM_020223.2 | dentin matrix protein 4 [Homo |
| | | | sapiens] |
| 574 | gi\|187761323 | NM_001127211.1 | shootin-1 isoform a [Homo |
| | | | sapiens] |
| 575 | gi\|38372934 | NM_021948.3 | brevican core protein isoform |
| | | | 1 [Homo sapiens] |
| 576 | gi\|150378519 | NM_032530.1 | zinc finger protein 594 [Homo |
| | | | sapiens] |
| 577 | gi\|81174548 | NM_001037163.1 | STAT3-interacting protein as |
| | | | a repressor [Homo sapiens] |
| 578 | gi\|83921601 | NM_001037984.1 | putative sodium-coupled |
| | | | neutral amino acid |
| | | | transporter 10 isoform a |
| | | | [Homo sapiens] |
| 579 | gi\|222831659 | NM_198475.2 | hypothetical protein |
| | | | LOC284069 precursor [Homo |
| | | | sapiens] |
| 580 | gi\|46519152 | NM_020690.4 | ANKHD1-EIF4EBP3 protein [Homo |
| | | | sapiens] |
| 581 | gi\|166362736 | NM_001114134.1 | erythrocyte membrane protein |
| | | | band 4.2 isoform 2 [Homo |
| | | | sapiens] |
| 582 | gi\|153266933 | NM_002082.3 | G protein-coupled receptor |
| | | | kinase 6 isoform B [Homo |
| | | | sapiens] |
| 583 | gi\|89903006 | NM_002084.3 | glutathione peroxidase 3 |
| | | | precursor [Homo sapiens] |
| 584 | gi\|161353442 | NM_004550.4 | NADH dehydrogenase |
| | | | [ubiquinone] iron-sulfur |
| | | | protein 2, mitochondrial |
| | | | isoform 1 precursor [Homo |
| | | | sapiens] |
| 585 | gi\|23110924 | NM_002798.1 | proteasome subunit beta type- |
| | | | 6 precursor [Homo sapiens] |
| 586 | gi\|75750485 | NM_004773.2 | zinc finger HIT domain- |
| | | | containing protein 3 [Homo |
| | | | sapiens] |
| 587 | gi\|215422360 | NM_004786.2 | thioredoxin-like protein 1 |
| | | | [Homo sapiens] |
| 588 | gi\|156071502 | NM_006694.3 | protein JTB precursor [Homo |
| | | | sapiens] |
| 589 | gi\|6996927 | NM_006792.2 | mortality factor 4 [Homo |
| | | | sapiens] |
| 590 | gi\|28373191 | NM_007002.2 | proteasomal ubiquitin |
| | | | receptor ADRM1 precursor |
| | | | [Homo sapiens] |
| 591 | gi\|33188444 | NM_012090.3 | microtubule-actin cross- |
| | | | linking factor 1 isoform a |
| | | | [Homo sapiens] |
| 592 | gi\|34147578 | NM_014338.3 | phosphatidylserine |
| | | | decarboxylase proenzyme [Homo |
| | | | sapiens] |
| 593 | gi\|24308114 | NM_015649.1 | interferon regulatory factor |
| | | | 2-binding protein 1 [Homo |
| | | | sapiens] |
| 594 | gi\|115430234 | NM_001048201.1 | E3 ubiquitin-protein ligase |
| | | | UHRF1 isoform 1 [Homo |
| | | | sapiens] |
| 595 | gi\|56676370 | NM_013291.2 | cleavage and polyadenylation |
| | | | specificity factor subunit 1 |
| | | | [Homo sapiens] |
| 596 | gi\|7706711 | NM_016538.1 | NAD-dependent deacetylase |
| | | | sirtuin-7 [Homo sapiens] |
| 597 | gi\|16554603 | NM_016070.2 | 28S ribosomal protein S23, |
| | | | mitochondrial [Homo sapiens] |
| 598 | gi\|210147465 | NM_022164.2 | tubulointerstitial nephritis |
| | | | antigen-like isoform 1 |
| | | | precursor [Homo sapiens] |
| 599 | gi\|282400941 | NM_022737.2 | lipid phosphate phosphatase- |
| | | | related protein type 2 |
| | | | isoform 1 [Homo sapiens] |
| 600 | gi\|154426254 | NM_032361.2 | THO complex subunit 3 [Homo |
| | | | sapiens] |
| 601 | gi\|34304353 | NM_183244.1 | phosphatase and actin |
| | | | regulator 3 isoform 2 [Homo |
| | | | sapiens] |
| 602 | gi\|194380673 | AK295603.1 | unnamed protein product [Homo |
| | | | sapiens] |
| 603 | gi\|66882523 | NM_001018676.1 | protein |
| | | | farnesyltransferase/geranylgeranyltransferase |
| | | | type-1 |
| | | | subunit alpha isoform b [Homo |
| | | | sapiens] |
| 604 | gi\|38788318 | NM_005275.2 | guanine nucleotide-binding |
| | | | protein-like 1 [Homo sapiens] |
| 605 | gi\|194688131 | NM_002129.3 | high mobility group protein |
| | | | B2 [Homo sapiens] |
| 606 | gi\|156119624 | NM_002215.2 | inter-alpha-trypsin inhibitor |
| | | | heavy chain H1 isoform a |
| | | | [Homo sapiens] |
| 607 | gi\|167614503 | NM_002291.2 | laminin subunit beta-1 |
| | | | precursor [Homo sapiens] |
| 608 | gi\|205277382 | NM_020998.3 | hepatocyte growth factor-like |
| | | | protein precursor [Homo |
| | | | sapiens] |
| 609 | gi\|24430150 | NM_002802.2 | 26S protease regulatory |
| | | | subunit 4 [Homo sapiens] |
| 610 | gi\|5803186 | NM_006755.1 | transaldolase [Homo sapiens] |
| 611 | gi\|31377804 | NM_003425.2 | zinc finger protein 45 [Homo |
| | | | sapiens] |
| 612 | gi\|62739174 | NM_003610.3 | mRNA export factor [Homo |
| | | | sapiens] |
| 613 | gi\|109452590 | NM_003849.2 | succinyl-CoA ligase [GDP- |
| | | | forming] subunit alpha, |
| | | | mitochondrial precursor [Homo |
| | | | sapiens] |
| 614 | gi\|187960106 | NM_003910.3 | protein BUD31 homolog [Homo |
| | | | sapiens] |
| 615 | gi\|117938253 | NM_001077441.1 | bcl-2-associated |
| | | | transcription factor 1 |
| | | | isoform 3 [Homo sapiens] |
| 616 | gi\|86788141 | NM_130442.2 | engulfment and cell motility |
| | | | protein 1 isoform 2 [Homo |
| | | | sapiens] |
| 617 | gi\|260656006 | NM_021102.3 | kunitz-type protease |
| | | | inhibitor 2 isoform a |
| | | | precursor [Homo sapiens] |
| 618 | gi\|4884170 | AL049924.1 | hypothetical protein [Homo |
| | | | sapiens] |
| 619 | gi\|194385125 | AK298842.1 | unnamed protein product [Homo |
| | | | sapiens] |
| 620 | gi\|41352716 | NM_015902.4 | E3 ubiquitin-protein ligase |
| | | | UBR5 [Homo sapiens] |
| 621 | gi\|22035561 | NM_018310.2 | transcription factor IIIB 50 kDa |
| | | | subunit [Homo sapiens] |
| 622 | gi\|122939166 | NM_019613.3 | WD repeat domain |
| | | | phosphoinositide-interacting |
| | | | protein 3 [Homo sapiens] |
| 623 | gi\|151301034 | NM_020151.3 | stAR-related lipid transfer |
| | | | protein 7, mitochondrial |
| | | | precursor [Homo sapiens] |
| 624 | gi\|20127622 | NM_024102.2 | methylosome protein 50 [Homo |
| | | | sapiens] |
| 625 | gi\|75677352 | NM_031921.4 | ATPase family AAA domain- |
| | | | containing protein 3B [Homo |
| | | | sapiens] |
| 626 | gi\|221139702 | NM_031925.2 | transmembrane protein 120A |
| | | | [Homo sapiens] |
| 627 | gi\|238550193 | NM_032298.2 | synaptotagmin-3 [Homo |
| | | | sapiens] |
| 628 | gi\|226437633 | NM_032590.4 | lysine-specific demethylase |
| | | | 2B isoform a [Homo sapiens] |
| 629 | gi\|201025403 | NM_174908.3 | coiled-coil domain-containing |
| | | | protein 50 short isoform |
| | | | [Homo sapiens] |
| 630 | gi\|34335395 | NM_032515.3 | bcl-2-related ovarian killer |
| | | | protein [Homo sapiens] |
| 631 | gi\|28872801 | NM_001212.3 | complement component 1 Q |
| | | | subcomponent-binding protein, |
| | | | mitochondrial precursor [Homo |
| | | | sapiens] |
| 632 | gi\|189339250 | NM_000387.4 | mitochondrial |
| | | | carnitine/acylcarnitine |
| | | | carrier protein [Homo |
| | | | sapiens] |
| 633 | gi\|149999367 | NM_004394.2 | death-associated protein 1 |
| | | | [Homo sapiens] |
| 634 | gi\|55956920 | NM_004499.3 | heterogeneous nuclear |
| | | | ribonucleoprotein A/B isoform |
| | | | b [Homo sapiens] |
| 635 | gi\|62632770 | NM_001015054.1 | DNA-3-methyladenine |
| | | | glycosylase isoform c [Homo |
| | | | sapiens] |
| 636 | gi\|76150622 | NM_006170.2 | putative ribosomal RNA |
| | | | methyltransferase NOP2 [Homo |
| | | | sapiens] |
| 637 | gi\|33239449 | NM_002592.2 | proliferating cell nuclear |
| | | | antigen [Homo sapiens] |
| 638 | gi\|77539056 | NM_003333.3 | ubiquitin-60S ribosomal |
| | | | protein L40 precursor [Homo |
| | | | sapiens] |
| 639 | gi\|190684674 | NM_006297.2 | DNA repair protein XRCC1 |
| | | | [Homo sapiens] |
| 640 | gi\|52630422 | NM_003581.2 | cytoplasmic protein NCK2 |
| | | | isoform A [Homo sapiens] |
| 641 | gi\|197276633 | NM_003707.2 | ruvB-like 1 [Homo sapiens] |
| 642 | gi\|226958573 | NM_004841.3 | ras GTPase-activating protein |
| | | | nGAP isoform 1 [Homo sapiens] |
| 643 | gi\|50428923 | NM_004854.3 | carbohydrate sulfotransferase |
| | | | 10 [Homo sapiens] |
| 644 | gi\|259013555 | NM_004860.3 | fragile X mental retardation |
| | | | syndrome-related protein 2 |
| | | | [Homo sapiens] |
| 645 | gi\|53729351 | NM_005112.4 | WD repeat-containing protein |
| | | | 1 isoform 2 [Homo sapiens] |
| 646 | gi\|6807758 | AL137297.1 | hypothetical protein [Homo |
| | | | sapiens] |
| 647 | gi\|14149701 | NM_015528.1 | E3 ubiquitin-protein ligase |
| | | | RNF167 precursor [Homo |
| | | | sapiens] |
| 648 | gi\|24430138 | NM_015540.2 | RNA polymerase II-associated |
| | | | protein 1 [Homo sapiens] |
| 649 | gi\|187829711 | NM_014417.3 | bcl-2-binding component 3 |
| | | | isoform 4 [Homo sapiens] |
| 650 | gi\|22547135 | NM_015956.2 | 39S ribosomal protein L4, |
| | | | mitochondrial isoform a [Homo |
| | | | sapiens] |
| 651 | gi\|187936926 | NM_014306.4 | tRNA-splicing ligase RtcB |
| | | | homolog [Homo sapiens] |
| 652 | gi\|116063563 | NM_018218.2 | ubiquitin carboxyl-terminal |
| | | | hydrolase 40 [Homo sapiens] |
| 653 | gi\|260166617 | NM_018127.6 | zinc phosphodiesterase ELAC |
| | | | protein 2 isoform 1 [Homo |
| | | | sapiens] |
| 654 | gi\|242247074 | NM_001009877.2 | bromodomain-containing |
| | | | protein 9 isoform 2 [Homo |
| | | | sapiens] |
| 655 | gi\|110349768 | NM_024296.3 | coiled-coil domain-containing |
| | | | protein 28B [Homo sapiens] |
| 656 | gi\|227500152 | NM_024333.2 | fibronectin type III and SPRY |
| | | | domain-containing protein 1 |
| | | | [Homo sapiens] |
| 657 | gi\|33337995 | AF173977.1 | MSTP107 [Homo sapiens] |
| 658 | gi\|31377666 | NM_031490.2 | lon protease homolog 2, |
| | | | peroxisomal [Homo sapiens] |
| 659 | gi\|206597512 | NM_015242.4 | arf-GAP with Rho-GAP domain, |
| | | | ANK repeat and PH domain- |
| | | | containing protein 1 isoform |
| | | | a [Homo sapiens] |
| 660 | gi\|45387954 | NM_205767.1 | protein QIL1 [Homo sapiens] |
| 661 | gi\|148596939 | NM_152383.4 | DIS3-like exonuclease 2 [Homo |
| | | | sapiens] |
| 662 | gi\|42794621 | NM_203374.1 | zinc finger protein 784 [Homo |
| | | | sapiens] |
| 663 | gi\|113424118 | XM_370711.5 | PREDICTED: similar to FRAS1 |
| | | | related extracellular matrix |
| | | | protein 2 [Homo sapiens] |
| 664 | gi\|45446741 | NM_001354.4 | aldo-keto reductase family 1 |
| | | | member C2 isoform 1 [Homo |
| | | | sapiens] |
| 665 | gi\|100913205 | NM_001357.3 | ATP-dependent RNA helicase A |
| | | | [Homo sapiens] |
| 666 | gi\|215276985 | NM_145740.3 | glutathione S-transferase A1 |
| | | | [Homo sapiens] |
| 667 | gi\|31657145 | NM_033453.2 | inosine triphosphate |
| | | | pyrophosphatase isoform a |
| | | | [Homo sapiens] |
| 668 | gi\|89276761 | NM_032940.2 | DNA-directed RNA polymerase |
| | | | II subunit RPB3 [Homo |
| | | | sapiens] |
| 669 | gi\|110618252 | NM_005035.3 | DNA-directed RNA polymerase, |
| | | | mitochondrial precursor [Homo |
| | | | sapiens] |
| 670 | gi\|42794609 | NM_002939.3 | ribonuclease inhibitor [Homo |
| | | | sapiens] |
| 671 | gi\|157266329 | NM_001033.3 | ribonucleoside-diphosphate |
| | | | reductase large subunit [Homo |
| | | | sapiens] |
| 672 | gi\|71051615 | NM_000374.3 | uroporphyrinogen |
| | | | decarboxylase [Homo sapiens] |
| 673 | gi\|45269143 | NM_012289.3 | kelch-like ECH-associated |
| | | | protein 1 [Homo sapiens] |
| 674 | gi\|194294518 | NM_001130102.1 | oxysterols receptor LXR-alpha |
| | | | isoform c [Homo sapiens] |
| 675 | gi\|7706336 | NM_016057.1 | coatomer subunit zeta-1 [Homo |
| | | | sapiens] |
| 676 | gi\|122114657 | NM_015037.2 | hypothetical protein LOC23053 |
| | | | isoform 1 [Homo sapiens] |
| 677 | gi\|150456431 | NM_001099436.1 | serine/threonine-protein |
| | | | kinase ULK3 [Homo sapiens] |
| 678 | gi\|118572609 | NM_016183.3 | mRNA turnover protein 4 |
| | | | homolog [Homo sapiens] |
| 679 | gi\|116812574 | NM_016258.2 | YTH domain family protein 2 |
| | | | isoform 1 [Homo sapiens] |
| 680 | gi\|21327682 | NM_032630.2 | cyclin-dependent kinase 2- |
| | | | interacting protein isoform 2 |
| | | | [Homo sapiens] |
| 681 | gi\|62526025 | NM_001014840.1 | protein CutA isoform 3 [Homo |
| | | | sapiens] |
| 682 | gi\|68533248 | NM_018476.3 | protein BEX1 [Homo sapiens] |
| 683 | gi\|94818878 | NM_001040457.1 | rhomboid domain-containing |
| | | | protein 2 isoform b [Homo |
| | | | sapiens] |
| 684 | gi\|83779009 | NM_024077.3 | selenocysteine insertion |
| | | | sequence-binding protein 2 |
| | | | [Homo sapiens] |
| 685 | gi\|208431768 | NM_031450.3 | basophilic leukemia expressed |
| | | | protein BLES03 isoform 2 |
| | | | [Homo sapiens] |
| 686 | gi\|32305518 | NM_031492.2 | RNA-binding protein 4B [Homo |
| | | | sapiens] |
| 687 | gi\|40317613 | NM_138777.2 | ribosome-recycling factor, |
| | | | mitochondrial isoform 1 |
| | | | precursor [Homo sapiens] |
| 688 | gi\|194473692 | NM_138340.4 | abhydrolase domain-containing |
| | | | protein 3 [Homo sapiens] |
| 689 | gi\|34304361 | NM_015102.2 | nephrocystin-4 [Homo sapiens] |
| 690 | gi\|49169840 | NM_001001795.1 | hypothetical protein |
| | | | LOC414919 [Homo sapiens] |
| 691 | gi\|89276783 | NM_001617.2 | beta-adducin isoform a [Homo |
| | | | sapiens] |
| 692 | gi\|148539875 | NM_001619.3 | beta-adrenergic receptor |
| | | | kinase 1 [Homo sapiens] |
| 693 | gi\|33591068 | NM_000386.2 | bleomycin hydrolase [Homo |
| | | | sapiens] |
| 694 | gi\|55770843 | NM_001903.2 | catenin alpha-1 [Homo |
| | | | sapiens] |
| 695 | gi\|40288196 | NM_005257.3 | transcription factor GATA-6 |
| | | | [Homo sapiens] |
| 696 | gi\|85838503 | NM_005318.3 | histone H1.0 [Homo sapiens] |
| 697 | gi\|194294499 | NM_005341.2 | zinc finger and BTB domain- |
| | | | containing protein 48 [Homo |
| | | | sapiens] |
| 698 | gi\|170014687 | NM_000414.2 | peroxisomal multifunctional |
| | | | enzyme type 2 isoform 2 [Homo |
| | | | sapiens] |
| 699 | gi\|33356546 | NM_004526.2 | DNA replication licensing |
| | | | factor MCM2 [Homo sapiens] |
| 700 | gi\|29826281 | NM_177983.1 | protein phosphatase 1G [Homo |
| | | | sapiens] |
| 701 | gi\|68216257 | NM_000981.3 | 60S ribosomal protein L19 |
| | | | [Homo sapiens] |
| 702 | gi\|197209833 | NM_005066.2 | splicing factor, proline- and |
| | | | glutamine-rich [Homo sapiens] |
| 703 | gi\|86991437 | NM_001039465.1 | serine/arginine-rich splicing |
| | | | factor 5 [Homo sapiens] |
| 704 | gi\|170014702 | NM_003023.4 | SH3 domain-binding protein 2 |
| | | | isoform a [Homo sapiens] |
| 705 | gi\|40806158 | NM_003250.4 | thyroid hormone receptor |
| | | | alpha isoform 2 [Homo |
| | | | sapiens] |
| 706 | gi\|31543803 | NM_006528.2 | tissue factor pathway |
| | | | inhibitor 2 precursor [Homo |
| | | | sapiens] |
| 707 | gi\|219842276 | NM_005439.2 | myeloid leukemia factor 2 |
| | | | [Homo sapiens] |
| 708 | gi\|201860299 | NM_003977.2 | AH receptor-interacting |
| | | | protein [Homo sapiens] |
| 709 | gi\|209862763 | NM_014806.2 | iporin [Homo sapiens] |
| 710 | gi\|124378038 | NM_014866.1 | protein transport protein |
| | | | Sec16A [Homo sapiens] |
| 711 | gi\|31652256 | NM_005461.3 | transcription factor MafB |
| | | | [Homo sapiens] |
| 712 | gi\|32307182 | NM_006379.2 | semaphorin-3C precursor [Homo |
| | | | sapiens] |
| 713 | gi\|32454736 | NM_033278.2 | tripartite motif-containing |
| | | | protein 3 [Homo sapiens] |
| 714 | gi\|260656035 | NM_006702.4 | neuropathy target esterase |
| | | | isoform b [Homo sapiens] |
| 715 | gi\|21758255 | AK098275.1 | unnamed protein product [Homo |
| | | | sapiens] |
| 716 | gi\|197313761 | NM_012137.3 | N(G),N(G)-dimethylarginine |
| | | | dimethylaminohydrolase 1 |
| | | | isoform 1 [Homo sapiens] |
| 717 | gi\|223468619 | NM_013446.3 | E3 ubiquitin-protein ligase |
| | | | makorin-1 isoform 1 [Homo |
| | | | sapiens] |
| 718 | gi\|11385647 | AF273045.1 | CTCL tumor antigen sel4-3 |
| | | | [Homo sapiens] |
| 719 | gi\|196115157 | NM_001131018.1 | cip1-interacting zinc finger |
| | | | protein isoform 4 [Homo |
| | | | sapiens] |
| 720 | gi\|55953121 | NM_015705.4 | small G protein signaling |
| | | | modulator 3 [Homo sapiens] |
| 721 | gi\|7020524 | AK000437.1 | unnamed protein product [Homo |
| | | | sapiens] |
| 722 | gi\|217035104 | NM_018198.3 | dnaJ homolog subfamily C |
| | | | member 11 [Homo sapiens] |
| 723 | gi\|34147349 | NM_024042.2 | meteorin precursor [Homo |
| | | | sapiens] |
| 724 | gi\|117320542 | NM_001077497.1 | proline-rich protein 3 |
| | | | isoform b [Homo sapiens] |
| 725 | gi\|113204623 | NM_032193.3 | ribonuclease H2 subunit C |
| | | | [Homo sapiens] |
| 726 | gi\|20270302 | NM_138769.1 | mitochondrial Rho GTPase 2 |
| | | | [Homo sapiens] |
| 727 | gi\|16445355 | NM_033415.2 | armadillo repeat containing |
| | | | protein 6 isoform 2 [Homo |
| | | | sapiens] |
| 728 | gi\|62244043 | NM_001014446.1 | OCIA domain-containing |
| | | | protein 2 isoform 1 [Homo |
| | | | sapiens] |
| 729 | gi\|333609247 | NM_153269.2 | hypothetical protein |
| | | | LOC140680 isoform 1 [Homo |
| | | | sapiens] |
| 730 | gi\|21389514 | NM_144726.1 | RING finger protein 145 |
| | | | isoform 2 [Homo sapiens] |
| 731 | gi\|197276595 | NM_006129.3 | bone morphogenetic protein 1 |
| | | | isoform 3 precursor [Homo |
| | | | sapiens] |
| 732 | gi\|169790898 | NM_001122630.1 | cyclin-dependent kinase |
| | | | inhibitor 1C isoform b [Homo |
| | | | sapiens] |
| 733 | gi\|66346646 | NM_001908.3 | cathepsin B preproprotein |
| | | | [Homo sapiens] |
| 734 | gi\|38201713 | NM_001419.2 | ELAV-like protein 1 [Homo |
| | | | sapiens] |
| 735 | gi\|46411186 | NM_005234.3 | nuclear receptor subfamily 2 |
| | | | group F member 6 [Homo |
| | | | sapiens] |
| 736 | gi\|167000329 | NM_021903.2 | gamma-aminobutyric acid type |
| | | | B receptor subunit 1 isoform |
| | | | b precursor [Homo sapiens] |
| 737 | gi\|27502382 | NM_172316.1 | homeobox protein Meis2 |
| | | | isoform h [Homo sapiens] |
| 738 | gi\|51317374 | NM_005008.2 | NHP2-like protein 1 [Homo |
| | | | sapiens] |
| 739 | gi\|20072756 | BC027470.1 | PCDHGC3 protein [Homo |
| | | | sapiens] |
| 740 | gi\|25777611 | NM_002809.2 | 26S proteasome non-ATPase |
| | | | regulatory subunit 3 [Homo |
| | | | sapiens] |
| 741 | gi\|125987582 | NM_080840.2 | receptor-type tyrosine- |
| | | | protein phosphatase alpha |
| | | | isoform 2 precursor [Homo |
| | | | sapiens] |
| 742 | gi\|21536317 | NM_006268.3 | zinc finger protein ubi-d4 |
| | | | [Homo sapiens] |
| 743 | gi\|194306565 | NM_007370.4 | replication factor C subunit |
| | | | 5 isoform 1 [Homo sapiens] |
| 744 | gi\|55925647 | NM_000994.3 | 60S ribosomal protein L32 |
| | | | [Homo sapiens] |
| 745 | gi\|42476326 | NM_003025.2 | endophilin-A2 isoform 1 [Homo |
| | | | sapiens] |
| 746 | gi\|56699493 | NM_003179.2 | synaptophysin [Homo sapiens] |
| 747 | gi\|83281440 | NM_003755.3 | eukaryotic translation |
| | | | initiation factor 3 subunit G |
| | | | [Homo sapiens] |
| 748 | gi\|331284175 | NM_001077261.3 | nuclear receptor corepressor |
| | | | 2 isoform 2 [Homo sapiens] |
| 749 | gi\|33519473 | NM_005831.3 | calcium-binding and coiled- |
| | | | coil domain-containing |
| | | | protein 2 [Homo sapiens] |
| 750 | gi\|40068460 | NM_007284.3 | twinfilin-2 [Homo sapiens] |
| 751 | gi\|19913459 | BC026067.1 | WDSOF1 protein [Homo sapiens] |
| 752 | gi\|155030235 | NM_001100412.1 | ribonuclease 3 isoform 2 |
| | | | [Homo sapiens] |
| 753 | gi\|46370087 | NM_13306.3 | sorting nexin-15 isoform A |
| | | | [Homo sapiens] |
| 754 | gi\|109134346 | NM_001042399.1 | coiled-coil domain-containing |
| | | | protein 41 [Homo sapiens] |
| 755 | gi\|50409706 | NM_016638.2 | ADP-ribosylation factor-like |
| | | | protein 6-interacting protein |
| | | | 4 isoform 2 [Homo sapiens] |
| 756 | gi\|19913437 | NM_015994.2 | V-type proton ATPase subunit |
| | | | D [Homo sapiens] |
| 757 | gi\|85362736 | NM_001039140.1 | hypothetical protein LOC54976 |
| | | | [Homo sapiens] |
| 758 | gi\|21750404 | AK091925.1 | unnamed protein product [Homo |
| | | | sapiens] |
| 759 | gi\|157388940 | NM_017998.2 | hypothetical protein LOC55071 |
| | | | [Homo sapiens] |
| 760 | gi\|189095252 | NM_001080425.2 | protein BEX4 [Homo sapiens] |
| 761 | gi\|46559760 | NM_032512.2 | PDZ domain-containing protein |
| | | | 4 [Homo sapiens] |
| 762 | gi\|198041727 | NM_001134774.1 | kinesin light chain 2 isoform |
| | | | 2 [Homo sapiens] |
| 763 | gi\|55749441 | NM_001006935.1 | transcription elongation |
| | | | factor A protein-like 4 [Homo |
| | | | sapiens] |
| 764 | gi\|18027791 | AF318350.1 | unknown [Homo sapiens] |
| 765 | gi\|116235475 | NM_032856.2 | WD repeat-containing protein |
| | | | 73 [Homo sapiens] |
| 766 | gi\|91176336 | NM_138383.2 | MTSS1-like protein [Homo |
| | | | sapiens] |
| 767 | gi\|156523245 | NM_145239.2 | proline-rich transmembrane |
| | | | protein 2 [Homo sapiens] |
| 768 | gi\|209571529 | NM_182527.2 | calcium-binding protein 7 |
| | | | [Homo sapiens] |
| 769 | gi\|195947381 | NM_178314.3 | RILP-like protein 1 [Homo |
| | | | sapiens] |
| 770 | gi\|34147601 | NM_004309.3 | rho GDP-dissociation |
| | | | inhibitor 1 isoform a [Homo |
| | | | sapiens] |
| 771 | gi\|23110949 | NM_001909.3 | cathepsin D preproprotein |
| | | | [Homo sapiens] |
| 772 | gi\|168480109 | NM_005225.2 | transcription factor E2F1 |
| | | | [Homo sapiens] |
| 773 | gi\|188497702 | NM_004186.3 | semaphorin-3F precursor [Homo |
| | | | sapiens] |
| 774 | gi\|109150415 | NM_012293.1 | peroxidasin homolog precursor |
| | | | [Homo sapiens] |
| 775 | gi\|115430228 | NM_005132.2 | meiotic recombination protein |
| | | | REC8 homolog [Homo sapiens] |
| 776 | gi\|24497575 | NM_006066.2 | alcohol dehydrogenase [NADP+] |
| | | | [Homo sapiens] |
| 777 | gi\|5453837 | NM_006396.1 | Sjoegren syndrome/scleroderma |
| | | | autoantigen 1 [Homo sapiens] |
| 778 | gi\|6005793 | NM_007213.1 | PRA1 family protein 2 [Homo |
| | | | sapiens] |
| 779 | gi\|209870093 | NM_012437.4 | SNARE-associated protein |
| | | | Snapin [Homo sapiens] |
| 780 | gi\|148664189 | NM_014333.3 | cell adhesion molecule 1 |
| | | | isoform 1 [Homo sapiens] |
| 781 | gi\|18496982 | NM_015526.1 | CAP-Gly domain-containing |
| | | | linker protein 3 [Homo |
| | | | sapiens] |
| 782 | gi\|238624145 | NM_017455.3 | neuroplastin isoform a |
| | | | precursor [Homo sapiens] |
| 783 | gi\|148833501 | NM_016403.3 | protein CWC15 homolog [Homo |
| | | | sapiens] |
| 784 | gi\|10438635 | AK025958.1 | unnamed protein product [Homo |
| | | | sapiens] |
| 785 | gi\|58761526 | NM_018181.4 | zinc finger protein 532 [Homo |
| | | | sapiens] |
| 786 | gi\|8922734 | NM_018255.1 | elongator complex protein 2 |
| | | | isoform 2 [Homo sapiens] |
| 787 | gi\|41327716 | NM_022104.3 | phosphorylated CTD- |
| | | | interacting factor 1 [Homo |
| | | | sapiens] |
| 788 | gi\|40068484 | NM_022834.3 | von Willebrand factor A |
| | | | domain-containing protein 1 |
| | | | isoform 1 precursor [Homo |
| | | | sapiens] |
| 789 | gi\|215820629 | NM_032355.3 | vacuolar fusion protein MON1 |
| | | | homolog A isoform a [Homo |
| | | | sapiens] |
| 790 | gi\|126090574 | NM_001039503.2 | serine protease 53 precursor |
| | | | [Homo sapiens] |
| 791 | gi\|24307878 | NM_001378.1 | cytoplasmic dynein 1 |
| | | | intermediate chain 2 [Homo |
| | | | sapiens] |
| 792 | gi\|153791534 | NM_021078.2 | histone acetyltransferase |
| | | | KAT2A [Homo sapiens] |
| 793 | gi\|73747843 | NM_002311.3 | DNA ligase 3 isoform beta |
| | | | precursor [Homo sapiens] |
| 794 | gi\|38045911 | NM_000269.2 | nucleoside diphosphate kinase |
| | | | A isoform b [Homo sapiens] |
| 795 | gi\|219879812 | NM_002696.2 | DNA-directed RNA polymerase |
| | | | II subunit RPB7 [Homo |
| | | | sapiens] |
| 796 | gi\|56790298 | NM_002779.3 | PH and SEC7 domain-containing |
| | | | protein 1 [Homo sapiens] |
| 797 | gi\|21359853 | NM_004582.2 | geranylgeranyl transferase |
| | | | type-2 subunit beta [Homo |
| | | | sapiens] |
| 798 | gi\|194394229 | NM_003313.3 | GDP-L-fucose synthase [Homo |
| | | | sapiens] |
| 799 | gi\|221136767 | NM_000371.3 | transthyretin precursor [Homo |
| | | | sapiens] |
| 800 | gi\|193211615 | NM_003634.2 | protein NipSnap homolog 1 |
| | | | isoform 1 [Homo sapiens] |
| 801 | gi\|21396488 | NM_004793.2 | lon protease homolog, |
| | | | mitochondrial precursor [Homo |
| | | | sapiens] |
| 802 | gi\|24234719 | NM_005494.2 | dnaJ homolog subfamily B |
| | | | member 6 isoform b [Homo |
| | | | sapiens] |
| 803 | gi\|86792514 | NM_005700.3 | dipeptidyl peptidase 3 [Homo |
| | | | sapiens] |
| 804 | gi\|34335235 | NM_006651.3 | complexin-1 [Homo sapiens] |
| 805 | gi\|116805324 | NM_170713.2 | ras association domain- |
| | | | containing protein 1 isoform |
| | | | C [Homo sapiens] |
| 806 | gi\|114796625 | NM_013356.2 | monocarboxylate transporter 3 |
| | | | [Homo sapiens] |
| 807 | gi\|95089415 | NM_012478.3 | WW domain-binding protein 2 |
| | | | [Homo sapiens] |
| 808 | gi\|51094102 | NM_019082.2 | probable ATP-dependent RNA |
| | | | helicase DDX56 [Homo sapiens] |
| 809 | gi\|33859677 | NM_017879.1 | zinc finger protein 416 [Homo |
| | | | sapiens] |
| 810 | gi\|24432025 | NM_032775.2 | kelch-like protein 22 [Homo |
| | | | sapiens] |
| 811 | gi\|126273571 | NM_144586.5 | ly6/PLAUR domain-containing |
| | | | protein 1 isoform a [Homo |
| | | | sapiens] |
| 812 | gi\|149363677 | NM_199287.2 | coiled-coil domain-containing |
| | | | protein 137 [Homo sapiens] |
| 813 | gi\|148763344 | NM_033529.2 | cyclin-dependent kinase 11A |
| | | | isoform 4 [Homo sapiens] |
| 814 | gi\|38372924 | NM_198589.1 | basigin isoform 2 precursor |
| | | | [Homo sapiens] |
| 815 | gi\|40804467 | NM_000723.3 | voltage-dependent L-type |
| | | | calcium channel subunit beta- |
| | | | 1 isoform 1 [Homo sapiens] |
| 816 | gi\|40549399 | NM_001894.4 | casein kinase I isoform |
| | | | epsilon [Homo sapiens] |
| 817 | gi\|34486089 | NM_004152.2 | ornithine decarboxylase |
| | | | antizyme 1 [Homo sapiens] |
| 818 | gi\|189458830 | NM_002880.3 | RAF proto-oncogene |
| | | | serine/threonine-protein |
| | | | kinase [Homo sapiens] |
| 819 | gi\|71164880 | NM_001011.3 | 40S ribosomal protein S7 |
| | | | [Homo sapiens] |
| 820 | gi\|71164876 | NM_001014.3 | 40S ribosomal protein S10 |
| | | | [Homo sapiens] |
| 821 | gi\|48255969 | NM_002969.3 | mitogen-activated protein |
| | | | kinase 12 [Homo sapiens] |
| 822 | gi\|56117849 | NM_007158.4 | cold shock domain-containing |
| | | | protein E1 isoform 2 [Homo |
| | | | sapiens] |
| 823 | gi\|149158693 | NM_080702.2 | large proline-rich protein |
| | | | BAG6 isoform b [Homo sapiens] |
| 824 | gi\|75992939 | NM_004651.3 | ubiquitin carboxyl-terminal |
| | | | hydrolase 11 [Homo sapiens] |
| 825 | gi\|156071485 | NM_003680.3 | tyrosyl-tRNA synthetase, |
| | | | cytoplasmic [Homo sapiens] |
| 826 | gi\|195972858 | NM_004228.5 | cytohesin-2 isoform 2 [Homo |
| | | | sapiens] |
| 827 | gi\|49472826 | NM_006324.2 | craniofacial development |
| | | | protein 1 [Homo sapiens] |
| 828 | gi\|61744459 | NM_017588.2 | WD repeat-containing protein |
| | | | 5 [Homo sapiens] |
| 829 | gi\|31543548 | NM_007273.3 | prohibitin-2 isoform 2 [Homo |
| | | | sapiens] |
| 830 | gi\|27374999 | NM_007285.6 | gamma-aminobutyric acid |
| | | | receptor-associated protein- |
| | | | like 2 [Homo sapiens] |
| 831 | gi\|38146101 | NM_013274.2 | DNA polymerase lambda isoform |
| | | | a [Homo sapiens] |
| 832 | gi\|171906572 | NM_014063.6 | drebrin-like protein isoform |
| | | | a [Homo sapiens] |
| 833 | gi\|17978480 | NM_080413.1 | vacuolar protein sorting- |
| | | | associated protein 16 homolog |
| | | | isoform 3 [Homo sapiens] |
| 834 | gi\|34147350 | NM_023940.2 | ras-like protein family |
| | | | member 11B [Homo sapiens] |
| 835 | gi\|190194415 | NM_024326.3 | F-box/LRR-repeat protein 15 |
| | | | [Homo sapiens] |
| 836 | gi\|16604251 | NM_052860.1 | zinc finger protein 300 |
| | | | isoform 2 [Homo sapiens] |
| 837 | gi\|115511029 | NM_138441.2 | protein MB21D1 [Homo sapiens] |
| 838 | gi\|23510412 | NM_052948.2 | rho GTPase-activating protein |
| | | | 33 isoform 1 [Homo sapiens] |
| 839 | gi\|223890145 | NM_182498.3 | zinc finger protein 428 [Homo |
| | | | sapiens] |
| 840 | gi\|58761495 | NM_001011724.1 | heterogeneous nuclear |
| | | | ribonucleoprotein A1-like 2 |
| | | | [Homo sapiens] |
| 841 | gi\|34530768 | AK124865.1 | unnamed protein product [Homo |
| | | | sapiens] |
| 842 | gi\|197100212 | NM_001610.2 | lysosomal acid phosphatase |
| | | | isoform 1 precursor [Homo |
| | | | sapiens] |
| 843 | gi\|34452697 | NM_004924.3 | alpha-actinin-4 [Homo |
| | | | sapiens] |
| 844 | gi\|17017985 | NM_001861.2 | cytochrome c oxidase subunit |
| | | | 4 isoform 1, mitochondrial |
| | | | precursor [Homo sapiens] |
| 845 | gi\|197245333 | NM_005273.3 | guanine nucleotide-binding |
| | | | protein G(I)/G(S)/G(T) |
| | | | subunit beta-2 [Homo sapiens] |
| 846 | gi\|21071025 | NM_005319.3 | histone H1.2 [Homo sapiens] |
| 847 | gi\|188497749 | NM_033500.2 | hexokinase-1 isoform HKI-td |
| | | | [Homo sapiens] |
| 848 | gi\|250083 | S37374.1 | HSJ1b [Homo sapiens] |
| 849 | gi\|219842228 | NM_004537.4 | nucleosome assembly protein |
| | | | 1-like 1 [Homo sapiens] |
| 850 | gi\|45439320 | NM_005038.2 | peptidyl-prolyl cis-trans |
| | | | isomerase D [Homo sapiens] |
| 851 | gi\|207029438 | NM_001135256.1 | histone-binding protein RBBP4 |
| | | | isoform c [Homo sapiens] |
| 852 | gi\|171543894 | NM_002973.3 | ataxin-2 [Homo sapiens] |
| 853 | gi\|51477701 | NM_001003801.1 | SWI/SNF-related matrix- |
| | | | associated actin-dependent |
| | | | regulator of chromatin |
| | | | subfamily D member 3 isoform |
| | | | 2 [Homo sapiens] |
| 854 | gi\|54607088 | NM_001005914.1 | semaphorin-3B isoform 2 |
| | | | precursor [Homo sapiens] |
| 855 | gi\|215820638 | NM_014714.3 | intraflagellar transport |
| | | | protein 140 homolog [Homo |
| | | | sapiens] |
| 856 | gi\|45643136 | NM_006791.2 | mortality factor 4-like |
| | | | protein 1 isoform 1 [Homo |
| | | | sapiens] |
| 857 | gi\|115511037 | NM_001009955.2 | single-stranded DNA-binding |
| | | | protein 3 isoform c [Homo |
| | | | sapiens] |
| 858 | gi\|194248096 | NM_013333.3 | epsin-1 isoform c [Homo |
| | | | sapiens] |
| 859 | gi\|223941871 | NM_017825.2 | poly(ADP-ribose) |
| | | | glycohydrolase ARH3 [Homo |
| | | | sapiens] |
| 860 | gi\|268370292 | NM_020442.4 | valyl-tRNA synthetase, |
| | | | mitochondrial isoform 2 |
| | | | precursor [Homo sapiens] |
| 861 | gi\|53831987 | NM_020659.2 | protein tweety homolog 1 |
| | | | isoform 1 [Homo sapiens] |
| 862 | gi\|165932369 | NM_024582.4 | protocadherin Fat 4 precursor |
| | | | [Homo sapiens] |
| 863 | gi\|52145308 | NM_032808.5 | leucine-rich repeat neuronal |
| | | | 6A [Homo sapiens] |
| 864 | gi\|150417982 | NM_001606.4 | ATP-binding cassette sub- |
| | | | family A member 2 isoform a |
| | | | [Homo sapiens] |
| 865 | gi\|71565153 | NM_000671.3 | alcohol dehydrogenase class-3 |
| | | | [Homo sapiens] |
| 866 | gi\|156523967 | NM_001618.3 | poly [ADP-ribose] polymerase |
| | | | 1 [Homo sapiens] |
| 867 | gi\|49249971 | NM_152296.3 | sodium/potassium-transporting |
| | | | ATPase subunit alpha-3 [Homo |
| | | | sapiens] |
| 868 | gi\|82546842 | NM_004327.3 | breakpoint cluster region |
| | | | protein isoform 1 [Homo |
| | | | sapiens] |
| 869 | gi\|20149593 | NM_007355.2 | heat shock protein HSP 90- |
| | | | beta [Homo sapiens] |
| 870 | gi\|27436949 | NM_005573.2 | lamin-B1 isoform 1 [Homo |
| | | | sapiens] |
| 871 | gi\|61742795 | NM_005581.3 | basal cell adhesion molecule |
| | | | isoform 1 precursor [Homo |
| | | | sapiens] |
| 872 | gi\|121256637 | NM_000918.3 | protein disulfide-isomerase |
| | | | precursor [Homo sapiens] |
| 873 | gi\|209529732 | NM_139032.2 | mitogen-activated protein |
| | | | kinase 7 isoform 2 [Homo |
| | | | sapiens] |
| 874 | gi\|257196241 | NM_001063.3 | serotransferrin precursor |
| | | | [Homo sapiens] |
| 875 | gi\|124028528 | NM_004819.2 | symplekin [Homo sapiens] |
| 876 | gi\|253735771 | NM_004723.3 | rho guanine nucleotide |
| | | | exchange factor 2 isoform 3 |
| | | | [Homo sapiens] |
| 877 | gi\|115298667 | NM_004814.2 | U5 small nuclear |
| | | | ribonucleoprotein 40 kDa |
| | | | protein [Homo sapiens] |
| 878 | gi\|148922919 | NM_014856.2 | DENN domain-containing |
| | | | protein 4B [Homo sapiens] |
| 879 | gi\|222080097 | NM_017450.2 | brain-specific angiogenesis |
| | | | inhibitor 1-associated |
| | | | protein 2 isoform 1 [Homo |
| | | | sapiens] |
| 880 | gi\|38455426 | NM_006430.2 | T-complex protein 1 subunit |
| | | | delta [Homo sapiens] |
| 881 | gi\|300192932 | NM_006796.2 | AFG3-like protein 2 [Homo |
| | | | sapiens] |
| 882 | gi\|166795249 | NM_006845.3 | kinesin-like protein KIF2C |
| | | | [Homo sapiens] |
| 883 | gi\|21396479 | NM_007367.2 | RNA-binding protein Raly |
| | | | short isoform [Homo sapiens] |
| 884 | gi\|150010638 | NM_015276.1 | ubiquitin carboxyl-terminal |
| | | | hydrolase 22 [Homo sapiens] |
| 885 | gi\|82659108 | NM_020765.2 | E3 ubiquitin-protein ligase |
| | | | UBR4 [Homo sapiens] |
| 886 | gi\|151101234 | NM_012461.2 | TERF1-interacting nuclear |
| | | | factor 2 isoform 2 [Homo |
| | | | sapiens] |
| 887 | gi\|134284354 | NM_012336.3 | nuclear prelamin A |
| | | | recognition factor isoform a |
| | | | [Homo sapiens] |
| 888 | gi\|50726984 | NM_016337.2 | ena/VASP-like protein [Homo |
| | | | sapiens] |
| 889 | gi\|37622893 | NM_194460.1 | RING finger protein 126 [Homo |
| | | | sapiens] |
| 890 | gi\|20521787 | AB033013.2 | KIAA1187 protein [Homo |
| | | | sapiens] |
| 891 | gi\|112789552 | NM_020820.3 | phosphatidylinositol 3,4,5- |
| | | | trisphosphate-dependent Rac |
| | | | exchanger 1 protein [Homo |
| | | | sapiens] |
| 892 | gi\|224549001 | NM_032905.4 | splicing factor 45 [Homo |
| | | | sapiens] |
| 893 | gi\|209862908 | NM_001136053.1 | transmembrane protein |
| | | | adipocyte-associated 1 |
| | | | isoform 1 [Homo sapiens] |
| 894 | gi\|197102773 | NM_007099.3 | low molecular weight |
| | | | phosphotyrosine protein |
| | | | phosphatase isoform b [Homo |
| | | | sapiens] |
| 895 | gi\|239937553 | NM_000687.2 | adenosylhomocysteinase |
| | | | isoform 1 [Homo sapiens] |
| 896 | gi\|18201904 | NM_000175.2 | glucose-6-phosphate isomerase |
| | | | isoform 2 [Homo sapiens] |
| 897 | gi\|169259765 | NM_001514.5 | transcription initiation |
| | | | factor IIB [Homo sapiens] |
| 898 | gi\|187761305 | NM_002134.3 | heme oxygenase 2 [Homo |
| | | | sapiens] |
| 899 | gi\|10434001 | AK022548.1 | unnamed protein product [Homo |
| | | | sapiens] |
| 900 | gi\|24797084 | NM_002265.4 | importin subunit beta-1 [Homo |
| | | | sapiens] |
| 901 | gi\|156631004 | NM_002812.4 | 26S proteasome non-ATPase |
| | | | regulatory subunit 8 [Homo |
| | | | sapiens] |
| 902 | gi\|154759258 | NM_003127.2 | spectrin alpha chain, brain |
| | | | isoform 2 [Homo sapiens] |
| 903 | gi\|38505154 | NM_003195.4 | transcription elongation |
| | | | factor A protein 2 isoform a |
| | | | [Homo sapiens] |
| 904 | gi\|141801911 | NM_003295.2 | translationally-controlled |
| | | | tumor protein [Homo sapiens] |
| 905 | gi\|21396499 | NM_003609.2 | HIRA-interacting protein 3 |
| | | | [Homo sapiens] |
| 906 | gi\|83367070 | NM_003753.3 | eukaryotic translation |
| | | | initiation factor 3 subunit D |
| | | | [Homo sapiens] |
| 907 | gi\|83367079 | NM_003801.3 | glycosylphosphatidylinositol |
| | | | anchor attachment 1 protein |
| | | | [Homo sapiens] |
| 908 | gi\|33469915 | NM_003906.3 | 80 kDa MCM3-associated |
| | | | protein [Homo sapiens] |
| 909 | gi\|52486264 | NM_006029.4 | paraneoplastic antigen Ma1 |
| | | | [Homo sapiens] |
| 910 | gi\|34365370 | BX641004.1 | hypothetical protein [Homo |
| | | | sapiens] |
| 911 | gi\|215490016 | NM_012286.2 | mortality factor 4-like |
| | | | protein 2 [Homo sapiens] |
| 912 | gi\|148727367 | NM_015695.2 | bromodomain and PHD finger- |
| | | | containing protein 3 [Homo |
| | | | sapiens] |
| 913 | gi\|193788635 | NM_032687.3 | cysteine and histidine-rich |
| | | | protein 1 isoform 2 [Homo |
| | | | sapiens] |
| 914 | gi\|50345274 | NM_016185.2 | hematological and |
| | | | neurological expressed 1 |
| | | | protein isoform 1 [Homo |
| | | | sapiens] |
| 915 | gi\|56788357 | NM_020243.4 | mitochondrial import receptor |
| | | | subunit TOM22 homolog [Homo |
| | | | sapiens] |
| 916 | gi\|32455272 | NM_006648.3 | serine/threonine-protein |
| | | | kinase WNK2 [Homo sapiens] |
| 917 | gi\|119964727 | NM_152783.3 | D-2-hydroxyglutarate |
| | | | dehydrogenase, mitochondrial |
| | | | precursor [Homo sapiens] |
| 918 | gi\|114520614 | NM_001954.4 | epithelial discoidin domain- |
| | | | containing receptor 1 isoform |
| | | | 1 precursor [Homo sapiens] |
| 919 | gi\|62241006 | NM_001888.2 | mu-crystallin homolog isoform |
| | | | 1 [Homo sapiens] |
| 920 | gi\|206725516 | NM_000801.3 | peptidyl-prolyl cis-trans |
| | | | isomerase FKBP1A isoform a |
| | | | [Homo sapiens] |
| 921 | gi\|4505488 | NM_002539.1 | ornithine decarboxylase [Homo |
| | | | sapiens] |
| 922 | gi\|38679891 | NM_006223.2 | peptidyl-prolyl cis-trans |
| | | | isomerase NIMA-interacting 4 |
| | | | isoform 1 [Homo sapiens] |
| 923 | gi\|194018536 | NM_002766.2 | phosphoribosyl pyrophosphate |
| | | | synthase-associated protein 1 |
| | | | [Homo sapiens] |
| 924 | gi\|78217389 | NM_001004.3 | 60S acidic ribosomal protein |
| | | | P2 [Homo sapiens] |
| 925 | gi\|197927095 | NM_001030.4 | 40S ribosomal protein S27 |
| | | | [Homo sapiens] |
| 926 | gi\|54792063 | NM_003352.4 | small ubiquitin-related |
| | | | modifier 1 isoform a |
| | | | precursor [Homo sapiens] |
| 927 | gi\|31083149 | NM_003502.2 | axin-1 isoform a [Homo |
| | | | sapiens] |
| 928 | gi\|153792767 | NM_021195.4 | claudin-6 [Homo sapiens] |
| 929 | gi\|151108508 | NM_014859.4 | rho GTPase-activating protein |
| | | | 44 [Homo sapiens] |
| 930 | gi\|22907051 | NM_006409.2 | actin-related protein 2/3 |
| | | | complex subunit 1A isoform 1 |
| | | | [Homo sapiens] |
| 931 | gi\|54112115 | NM_006802.2 | splicing factor 3A subunit 3 |
| | | | [Homo sapiens] |
| 932 | gi\|209413761 | NM_017586.2 | calcium channel flower |
| | | | homolog isoform a [Homo |
| | | | sapiens] |
| 933 | gi\|209954817 | NM_014921.4 | latrophilin-1 isoform 2 |
| | | | precursor [Homo sapiens] |
| 934 | gi\|14591905 | NM_012423.2 | 60S ribosomal protein L13a |
| | | | [Homo sapiens] |
| 935 | gi\|38570153 | NM_012279.2 | zinc finger protein 346 [Homo |
| | | | sapiens] |
| 936 | gi\|44680150 | NM_014335.2 | EP300-interacting inhibitor |
| | | | of differentiation 1 [Homo |
| | | | sapiens] |
| 937 | gi\|166197689 | NM_001114090.1 | ephexin-1 isoform 2 [Homo |
| | | | sapiens] |
| 938 | gi\|75677342 | NM_015544.2 | transmembrane protein 98 |
| | | | [Homo sapiens] |
| 939 | gi\|27414496 | NM_014153.2 | zinc finger CCCH domain- |
| | | | containing protein 7A [Homo |
| | | | sapiens] |
| 940 | gi\|8922866 | NM_018322.1 | hypothetical protein LOC55776 |
| | | | [Homo sapiens] |
| 941 | gi\|142383690 | NM_022756.4 | chromatin modification- |
| | | | related protein MEAF6 [Homo |
| | | | sapiens] |
| 942 | gi\|186928845 | NM_032476.3 | 28S ribosomal protein S6, |
| | | | mitochondrial [Homo sapiens] |
| 943 | gi\|41872402 | NM_201398.1 | FAD synthase isoform 2 [Homo |
| | | | sapiens] |
| 944 | gi\|197304706 | NM_032350.5 | hypothetical protein LOC84310 |
| | | | [Homo sapiens] |
| 945 | gi\|56786146 | NM_001801.2 | cysteine dioxygenase type 1 |
| | | | [Homo sapiens] |
| 946 | gi\|55750040 | NM_001940.3 | atrophin-1 [Homo sapiens] |
| 947 | gi\|194097322 | NM_004092.3 | enoyl-CoA hydratase, |
| | | | mitochondrial precursor [Homo |
| | | | sapiens] |
| 948 | gi\|160420313 | NM_001456.3 | filamin-A isoform 1 [Homo |
| | | | sapiens] |
| 949 | gi\|89276752 | NM_198252.2 | gelsolin isoform b [Homo |
| | | | sapiens] |
| 950 | gi\|49472820 | NM_001539.2 | dnaJ homolog subfamily A |
| | | | member 1 [Homo sapiens] |
| 951 | gi\|62388891 | NM_002266.2 | importin subunit alpha-2 |
| | | | [Homo sapiens] |
| 952 | gi\|194440683 | NM_002337.2 | alpha-2-macroglobulin |
| | | | receptor-associated protein |
| | | | precursor [Homo sapiens] |
| 953 | gi\|93141028 | NM_001040134.1 | paralemmin isoform 2 [Homo |
| | | | sapiens] |
| 954 | gi\|103485495 | NM_002768.2 | charged multivesicular body |
| | | | protein 1a isoform 2 [Homo |
| | | | sapiens] |
| 955 | gi\|313760623 | NM_000442.4 | platelet endothelial cell |
| | | | adhesion molecule precursor |
| | | | [Homo sapiens] |
| 956 | gi\|47132588 | NM_002741.3 | serine/threonine-protein |
| | | | kinase N1 isoform 2 [Homo |
| | | | sapiens] |
| 957 | gi\|25777601 | NM_002808.3 | 26S proteasome non-ATPase |
| | | | regulatory subunit 2 [Homo |
| | | | sapiens] |
| 958 | gi\|30581139 | NM_006263.2 | proteasome activator complex |
| | | | subunit 1 isoform 1 [Homo |
| | | | sapiens] |
| 959 | gi\|104487294 | NM_130853.2 | receptor-type tyrosine- |
| | | | protein phosphatase S isoform |
| | | | 3 precursor [Homo sapiens] |
| 960 | gi\|71772428 | NM_001021.3 | 40S ribosomal protein S17 |
| | | | [Homo sapiens] |
| 961 | gi\|156416002 | NM_004168.2 | succinate dehydrogenase |
| | | | [ubiquinone] flavoprotein |
| | | | subunit, mitochondrial |
| | | | precursor [Homo sapiens] |
| 962 | gi\|118582263 | NM_006924.4 | serine/arginine-rich splicing |
| | | | factor 1 isoform 1 [Homo |
| | | | sapiens] |
| 963 | gi\|38181628 | BC061586.1 | TMSB4X protein [Homo sapiens] |
| 964 | gi\|37594440 | NM_003437.2 | zinc finger protein 136 [Homo |
| | | | sapiens] |
| 965 | gi\|83281439 | NM_003757.2 | eukaryotic translation |
| | | | initiation factor 3 subunit I |
| | | | [Homo sapiens] |
| 966 | gi\|23397695 | NM_152925.1 | copine-1 isoform a [Homo |
| | | | sapiens] |
| 967 | gi\|153791496 | NM_014675.3 | rootletin [Homo sapiens] |
| 968 | gi\|299829176 | NM_006989.5 | ras GTPase-activating protein |
| | | | 4 isoform 1 [Homo sapiens] |
| 969 | gi\|221307564 | NM_005805.4 | 26S proteasome non-ATPase |
| | | | regulatory subunit 14 [Homo |
| | | | sapiens] |
| 970 | gi\|138175804 | NM_006334.3 | noelin isoform 2 [Homo |
| | | | sapiens] |
| 971 | gi\|142379276 | NM_006541.3 | glutaredoxin-3 [Homo sapiens] |
| 972 | gi\|40805821 | NM_007263.3 | coatomer subunit epsilon |
| | | | isoform a [Homo sapiens] |
| 973 | gi\|254911095 | NM_001042486.2 | disks large-associated |
| | | | protein 4 isoform c [Homo |
| | | | sapiens] |
| 974 | gi\|194328807 | NM_012267.4 | hsp70-binding protein 1 [Homo |
| | | | sapiens] |
| 975 | gi\|42716281 | NM_013242.2 | transcription factor IIB |
| | | | [Homo sapiens] |
| 976 | gi\|54792141 | NM_019845.2 | protein reprimo [Homo |
| | | | sapiens] |
| 977 | gi\|209915598 | NM_032853.3 | PWWP domain-containing |
| | | | protein MUM1 [Homo sapiens] |
| 978 | gi\|50086623 | NM_033547.3 | integrator complex subunit 4 |
| | | | [Homo sapiens] |

1-16. (canceled)

17. A method for identifying marker sequences for gynaecological malignoma, characterised in that
a) marker sequence candidates for gynaecological malignoma are identified by bringing a support, on which at least 1,000 different proteins are immobilised, into contact with a serum sample from a patient with gynaecological malignoma and proteins that demonstrate an interaction with the serum are identified as marker sequence candidates, and
b) the interaction of one or more marker sequence candidates from a) with the serum from female patients with gynaecological malignoma is determined compared with
the interaction of the marker sequence candidate(s) from a) with the serum from female patients with benign changes and
the interaction of the marker sequence candidate(s) from a) with the serum of healthy control individuals, and
c) marker sequences are identified that demonstrate an interaction with the serum from female patients with gynaecological malignoma that is different compared with the interaction with the serum from female patients with benign changes and the serum from healthy control individuals.

18. The method according to claim 17, characterised in that, for the comparison, the data concerning the interaction are evaluated by means of statistical analysis.

19. A marker sequence for gynaecological malignoma obtained by a method according to claim 17, wherein the marker sequence is selected from the group consisting of sequences comprising SEQ ID NOS: 1-1467 and partial sequences of SEQ ID NOS: 1-1467 with at least 90%, preferably 95%, of the length of the sequences SEQ ID NOS: 1-1467 and homologues of SEQ ID NOS: 1-1467 and partial sequences thereof with an identity of at least 95%, preferably 98% or more, to the corresponding nucleic acid and/or protein sequences and sequences coded by SEQ ID NOS: 1-489, partial sequences thereof and homologues thereof.

20. An arrangement of marker sequences for gynaecological malignoma, comprising one or more marker sequences according to claim 19.

21. A protein array comprising one or more marker sequences according to claim 19.

22. A diagnostic tool comprising one or more marker sequences according to claim 19 and optionally further additives and/or excipients.

23. A test kit comprising one or more marker sequences according to claim 19 and optionally further additives and/or excipients.

24. The arrangement according to claim 20, wherein 2 or 3 different marker sequences for gynaecological malignoma are used simultaneously.

25. Use of one or more marker sequences according to claim 19 for the early detection, diagnosis, prognosis, therapy control and/or aftercare in the case of gynaecological malignoma.

26. Use of one or more marker sequences according to claim 19 to distinguish gynaecological malignoma from benign changes.

27. Use of one or more marker sequences according to claim 19 for the individualised diagnosis and/or therapy in individual patients, patient groups, cohorts, population groups, variants of gynaecological malignoma, or stages of gynaecological malignoma.

28. Use of one or more marker sequences according to claim 19 for the detection and/or for the determination of the quantity of one or more gynaecological malignoma-associated autoantibodies, for example in bodily fluid or tissue of a patient.

29. Use of one or more marker sequences according to claim 19 for the analysis of autoantibody profiles of patients, in particular for the qualitative and/or quantitative analysis of autoantibodies and/or for the monitoring of changes of autoantibody profiles, for example in bodily fluids such as serum, tissue or tissue samples from the patient.

30. Use of one or more marker sequences according to claim 19 for the screening of substances (active agents) for gynaecological malignoma.

31. A target for the treatment and/or therapy of gynaecological malignoma selected from one of the marker sequences according to claim 19.

32. A method for the early detection, diagnosis, prognosis, therapy control and/or aftercare in the case of gynaecological malignoma, wherein
a) a marker sequence or a number of marker sequences selected from the group comprising sequences SEQ ID NOS: 1-1467 and partial sequences of SEQ ID NOS: 1-1467 with at least 90%, preferably 95%, of the length of the sequences SEQ ID NOS: 1-1467 and homologues of SEQ ID NOS: 1-1467 and partial sequences thereof with an identity of at least 95%, preferably 98% or more, to the corresponding nucleic acid and/or protein sequences and sequences coded by SEQ ID NOS: 1-489, partial sequences thereof and homologues thereof is/are applied to a support,
b) the marker sequence or number of marker sequences is/are brought into contact with bodily fluid or tissue sample from a patient, and
c) an interaction of the bodily fluid or the tissue sample with the marker sequence(s) for gynaecological malignoma from a) is detected.
